(19) United States
(12) Patent Application Publication
(10) Pub. No.: US 2003/0008838 A1
(43) Pub. Date: Jan. 9, 2003

(54) Method of increasing endogenous leptin production

(76) Inventors: Peter J. Havel, Davis, CA (US); Joseph L. Evans, Palo Alto, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/114,335

(22) Filed: Apr. 1, 2002

Related U.S. Application Data
(60) Provisional application No. 60/281,285, filed on Apr. 3, 2001.

(51) Int. Cl. A61K 48/00; A61K 31/4015; A61K 31/195; A61K 31/19

(52) U.S. Cl. 514/44; 514/424; 514/562; 514/557; 435/455

(57) ABSTRACT

A formulation for and method of enhancing leptin secretion is disclosed. The method comprises contacting living cells with an inhibitor of the enzyme pyruvate dehydrogenase kinase (PDHK). The PDHK inhibitor causes the cells it contacts to increase leptin secretion. The increased levels of leptin provides a range of desired results including weight loss and the prevention of weight regain after weight loss resulting from dieting and/or exercise.

### CROSS REFERENCES

This application claims priority to earlier filed provisional application Serial No. 60/281,285 filed Apr. 3, 2001, which application is incorporated herein by reference in its entirety.

### GOVERNMENT RIGHTS

The United States Government may have creation rights in this application pursuant to Grant DK-50129 and DK-35747 from the National Institutes of Health.

### FIELD OF THE INVENTION

The invention relates generally to the field of pharmaceuticals and methods of treatment and more particularly to pharmaceutical formulations which inhibit pyruvate dehydrodegenase kinase or activate malic enzyme and methods of administering such formulations in a manner which enhances leptin production and/or secretion from cells.

### BACKGROUND OF THE INVENTION

Obesity is a serious and increasingly prevalent health problem, which is associated with an increased risk of type-2 diabetes, hyperlipidemia, and hypertension. The discovery of the adipocyte hormone, leptin, has dramatically impacted the field of obesity research. Leptin acts in the CNS to regulate food intake and energy expenditure, and in the periphery is involved in the regulation of metabolic substrate fluxes, including paracrine actions in adipose tissue itself. Normal leptin production and action are essential for maintaining energy balance. Humans and animals that cannot make leptin or respond to leptin due to receptor defects overeat and become markedly obese. Even partial leptin deficiency due to a heterozygous genetic defect in leptin production has been shown to lead to increased weight gain and adiposity (body fat content)(Farooqi et al, Nature, 2002). Circulating leptin concentrations are chronically regulated by adipose mass and acutely regulated by insulin responses to recent energy (food) intake (see Reviews, Havel, Am. J. Clin. Nutr., 1999, Proc. Nutr. Soc., 2000, Exp. Biol. Med., 2001, and Curr. Opin. Lipidol., 2002).

Increased sensations of hunger during dieting are related to decreases of circulating leptin during energy restriction (dieting) in humans (Keim et al, Am. J. Clin. Nutr., 1998) and decreased leptin production is likely to contribute to weight regain after weight loss achieved by dieting. Decreased leptin may also contribute to the fall of metabolic rate that occurs during energy-restricted diets (see Reviews, Havel, Proc. Nutr, Soc., 2000, Exp. Biol. Med., 2001). Therefore a method to stimulate endogenous leptin production (i.e. an agent that increases leptin production), in concert with dieting, could help in the induction and maintenance of weight loss by preventing leptin production and circulating leptin levels from falling.

Increasing endogenous leptin production represents a novel approach to the treatment of obesity which clearly differs from the current strategy of administering exogenous leptin of recombinant origin (Heymsfield et al, JAMA. 282: 1568-1575, 1999). Since leptin has a number of actions beyond the regulation of energy balance, in addition to obesity management, a method for increasing endogenous leptin production could be useful for modulating glucose and lipid metabolism, hypothalamic-pituitary neuroendocrine function, treatment of infertility, and to promote immune function, hematopoiesis, as well as to increase angiogenesis and wound healing. For example, leptin administration was recently shown to improve glucose control and decrease serum lipids (triglycerides) in humans with diabetes due to defects in fat deposition (lipodystrophy)(Oral et al, New Engl. J. Med., 2002). A major advantage of the endogenous approach is the potential that orally-available small molecule stimulators of leptin production could be found and/or designed. Small molecule agents are considerably less costly to produce and would avoid the problems associated with the pain of daily injections and the significant injection site reactions that have been reported with subcutaneous administration of recombinant leptin (Heymsfield et al, JAMA. 282: 1568-1575, 1999). Circulating leptin levels are regulated by insulin responses to meals. Data has been generated from experiments in cultured adipocytes in vitro that indicate that glucose utilization is an important determinant of insulin-mediated leptin gene expression and leptin secretion (Mueller et al, Endocrinology, 1998). We have also shown that anaerobic metabolism of glucose to lactate does not result in increased leptin secretion (Mueller et al, Obesity Res., 8:530-539, 2000). Additional information indicates a mechanism that requires increasing the transport of substrate into the mitochondria for oxidation in the TCA cycle as a metabolic pathway by which insulin-mediated glucose metabolism regulates leptin production (Havel et al, Obesity Res., Abstract, 1999).

An important mechanism in the action of insulin to increase the flux of glucose carbon into the mitochondria for oxidative metabolism is activation of pyruvate dehydrogenase (PDH). The activity of PDH is decreased when it is phosphorylated and increases when it is dephosphorylated. Insulin increases PDH by activating a PDH phosphatase enzyme (Taylor, 1973). Another enzyme pyruvate dehydrogenase kinase (PDHK) inhibits the activity of PDH by phosphorylating the PDH enzyme complex.

### SUMMARY OF THE INVENTION

A pharmaceutical formulation for and method of enhancing endogenous leptin production and/or secretion is disclosed. The method comprises administering a therapeutically effective amount of a formulation comprising a compound which inhibits pyruvate dehydrogenate kinase (PDHK) thereby contacting cells (e.g. in a living animal) with the PDHK inhibitor. The formulation of PDHK inhibitor is allowed to act on the cells for a sufficient period of time and under conditions such that endogenous leptin secretion by the cells is enhanced relative to the level of leptin secretion prior to treatment. The level of enhanced secretion may be any detectable level above the pretreatment level of the cells and/or individual being treated and not necessarily above the level of a normal cell or normal individual. Preferably the level of enhancement is 10% or more above the pretreatment level, more preferably 25% or more and still more preferably 100% more above the pretreatment level. The biochemical and molecular (antisense) enhancements of glucose oxidation via inhibition of PDHK reported here increase leptin production by 30-80%. The level of enhanced leptin secretion can be monitored and adjustments made in dosing of the PDHK inhibitor formulation based on the measured results obtained. The leptin level obtained by the treatment is preferably therapeutic in terms of obtaining a desired overall desired result or effect not only on a cell or group of cells but on an individual, e.g. obtain weight loss.

Obese individuals with relatively low leptin levels relative to normal individuals are likely to be most responsive to the treatments as provided here. Increasing the metabolic flux through pyruvate dehydrogenase (PDH) by inhibiting its regulatory enzyme PDH-kinase (PDHK) stimulates the production of adipocyte hormone leptin. The regulatory enzyme PDH-K can be effected in different ways. For example, antisense sequences to PDH-K disrupts PDH-K production which decreases anaerobic glucose metabolism and stimulates leptin production. In another example small molecules directly or indirectly inhibit the enzymatic activity of PDH-K which in turn stimulates leptin production. Both antisense and small molecule inhibitors can be used in combination to increase leptin production.

In addition to small molecule inhibitors of PDHK and the use of antisense it is possible to enhance endogenous leptin production by transfecting cells with malic enzyme. Any combination of two or three of these methods can be combined together to further enhance endogenous leptin production.

An aspect of the invention is a formulation comprising a therapeutically effective amount of a PDHK inhibitor and a pharmaceutically acceptable carrier preferably provided in a readily administrable dosage form useful in enhancing leptin secretion.

Another aspect of the invention is a pharmaceutical formulation comprising a carrier and a PDH-K inhibitor.

In a particular embodiment the formulation comprises an antisense sequence as the PDH-K inhibitor.

In another particular embodiment the formulation comprises an orally active small molecule PDH-K inhibitor as the active ingredient.

Another aspect of the invention is a method comprised of contacting cells with a formulation which inhibits the enzyme pyruvate dehydrogenase kinase (PDHK) in a manner which results in increasing leptin production.

An advantage of the invention is that enhanced leptin levels can be obtained without the administration of exogenous leptin.

Another advantage of the invention is that enhanced leptin levels provide desired effects including weight loss and preventing weight gain after successful weight loss from dieting and/or exercise.

An aspect of the invention is a method for treating obesity by stimulating endogenous leptin production (i.e., the use of pharmacological agents that increase leptin production by adipose tissue).

Another aspect of the invention is increasing leptin production to modulate glucose and lipid metabolism in diabetes and hyperlipidemia, hypothalamic-pituitary neuroendocrine function, treat infertility and to promote immune function, hematopoiesis, as well to increase angiogenesis and wound healing.

Yet another aspect of the invention is the development of new targets for compounds to accomplish the stimulation of leptin production by increasing the metabolic flux of carbon from glucose into oxidative metabolism in the TCA cycle through a pathway involving the enzyme pyruvate dehydrogenase (PDH) by inhibiting its regulatory enzyme PDH kinase, activating PDH phosphatase, or other pathways of adipocyte metabolism such as malic enzyme and lactate dehydrogenase.

Still another aspect of the invention is the use of specific inhibitors of PDH kinase or activators of PDH phosphatase which we have demonstrated increase glucose utilization, without stimulating anaerobic glucose metabolism into lactate, and increase leptin production from isolated cultured adipocytes.

Another aspect of the invention is the use of specific compounds to activate other metabolic pathways of adipocyte metabolism including, but not limited to, NADPH malic enzyme, lactate dehydrogenase, fatty acid oxidation, and/or cellular ATP (adenylate charge) and redox status (NADH/NAD and NADPH/NADP ratios) which are shown here to affect the regulation of leptin production by adipose tissue.

Another aspect of the invention is a method of treatment of individuals with abnormally low levels of leptin.

Another aspect of the invention is a formulation manufactured for the treatment of cells and/or individuals which do not produce sufficient levels of leptin.

These and other objects, advantages, and features of the invention will become apparent to those persons skilled in the art upon reading the details of the formulations and method as more fully described below.

### BRIEF DESCRIPTIONS OF THE DRAWINGS

FIG. 1 is a schematic diagram of events comparing the effects of metformin and insulin on aerobic vs. anaerobic glucose metabolism and leptin secretion in adipocytes.

FIG. 2 is a schematic diagram showing the flow of events involved in oxidative metabolism in adipocytes and the effects of agents (such as inhibitors of PDHK) which increase substrate oxidation and leptin production.

FIG. 3 is a schematic diagram showing events involved in pyruvate dehydrogenase regulation by insulin and PDH kinase inhibitors via their effects on PDH phosphatase and PDHK, respectively.

FIG. 4 is a schematic diagram showing events involved in the pyruvate-malate cycle and its activation by malate, fumarate and DCA.

FIGS. 5A and 5B are graphs that illustrate the effects of inhibition of translation (protein synthesis) using cycloheximide (cyclo; FIG. 5A) or transcription (gene expression) using actinomycin (actino; FIG. 5B) on basal and insulin-stimulated leptin production in isolated adipocytes.

FIG. 6 includes graphs 6A-6F which show the effects of Insulin on Adipocyte Metabolism and Leptin Secretion: Physiological levels of insulin (0.16 to 1.6 nM) induce concentration-dependent increases of leptin secretion from primary cultured adipocytes (FIG. 6A). Insulin also induces concentration-dependent (n=6 each) increases of glucose utilization (FIG. 6B), decreases the proportion of glucose converted to lactate (FIG. 6C), but does not affect the proportional incorporation of glucose into triglyceride (FIG. 6D). By subtraction, insulin increases the proportion of glucose that is not converted to lactate or incorporated into TG (OTHER)(FIG. 6E). The most likely fate of this glucose is mitochondrial oxidation and as shown in a separate experiment, insulin (1.6 nM) doubles the amount of glucose oxidized to CO₂ (FIG. 6F)(n=18) and increases the proportional oxidation of glucose oxidized by 43.6±10.6% (p<0.0025)(data not shown).

FIG. 7 includes graphs 7A-7D which show the effects of Anaerobic Metabolism: The anaerobic metabolism of glucose to lactate does not increase leptin production. This is illustrated by the inverse relationship between leptin from isolated adipocytes and the proportional conversion of glucose to lactate (FIG. 7A). In addition, metformin at a concentration of 1.0 mM (with 0.16 nM insulin) increases glucose utilization (FIG. 7B, p<0.0005), but increases the proportion of glucose metabolized to lactate (FIG. 7C, p<0.01), and inhibits leptin secretion (FIG. 7D, p<0.005) from isolated cultured adipocytes (n=18 each). Data are from Mueller, Obesity Res., 2000.

FIG. 8 includes graphs 8A-8D which show the effects of PDH Kinase Inhibitors on Adipocyte Metabolism and Leptin Secretion: The effects of three compounds, 5,5′-Dithiobis(2-nitrobenzoate)(DTNB)(n=11), N-ethylmaleimide (NEM)(n=6) and dichloroacetate (DCA)(n=6) that have been reported to act as inhibitors of PDH kinase were examined in isolated cultured adipocytes. All graphs compare the effects of the compounds as a percent of control values (* indicates p<0.05). DTNB and NEM, but not DCA, increased total glucose utilization by isolated adipocytes (FIG. 8A). All three compounds decreased the proportion of glucose that was anaerobically metabolized to lactate (FIG. 8B) DCA was the most effective. None of the three increased the proportion of glucose incorporated into TG (data not shown), but by subtraction increased the proportion (FIG. 8C), and the absolute amount of glucose (data not shown) that was not converted to lactate or incorporated into TG (OTHER)(FIG. 8C). All three compounds significantly increased leptin secretion by 30-60% (FIG. 8D). DTNB was the most potent stimulator of leptin production.

FIG. 9 includes graphs 9A-9D which show the effects of uncoupling oxidative phosphorylation with dinitrophenol and ATP measurement: The effects of the uncoupling agent, DNP, were investigated in isolated, cultured adipocytes. DNP (n=12) increased glucose utilization (FIG. 9A, p<0.0001). DNP increased both glucose (FIG. 9B, p<0.01) and fatty acid (FIG. 9C, p<0.01) oxidation (n=9 each)(shown as percent of control). DNP also modestly, but significantly (p<0.005) increased leptin secretion (n=12).

FIG. 10 includes the graphs 10A-10B which show the effects of Increasing Fatty Acid Oxidation with L-Camitine on Adipocyte Metabolism and Leptin Secretion: Camitine is a cofactor for the transport of fatty acids into the mitochondria for oxidation by camitine-palmitoyl-transferase (CPT). Camitine at a concentration of 20 mM (n=7) increased fatty acid oxidation (shown as percent of control) by 50% (p<0.02). Camitine also inhibited glucose utilization (p<0.0001), increased the proportion of glucose metabolized to lactate (p<0.0001), and inhibited the incorporation of labeled glucose into TG (p<0.0025)(data not shown). Despite these effects on glucose metabolism that would be expected to inhibit leptin production, leptin secretion was modestly increased (FIG. 6B, p<0.05, n=12), suggesting that, under these conditions (i.e. uncoupling), increased fatty acid oxidation can increase leptin production when glucose oxidation is suppressed.

FIG. 11 includes graphs 11A and 11B which show the effects of Malate, Fumarate, and Oleic Acid on Leptin Secretion: Malate (2-5 mM), the substrate for malic enzyme, or fumarate (5 mM), an allosteric activator of malic enzyme, each increased leptin secretion by 20% (FIG. 11A, n=6 each, p<0.02). Malate and fumarate in combination produced a larger increase (45±7%, p<0.0025) of leptin secretion than either alone (FIG. 11A). The long-chain fatty acid, oleate (n=7) at a concentration of 2 mM, inhibited glucose oxidation by ˜30% (p<0.001) and increased leptin secretion (FIG. 11B, p<0.005) further suggesting that lipid oxidation can stimulate leptin production.

FIG. 12 includes graphs 12A and 12B which show data of the Regulation of the Leptin Promoter by Insulin and Glucose Metabolism: 3T3-L1 cells were transfected with a luciferase construct of the leptin promoter. Insulin at concentrations of 0.1 to 10 nM increased the activity of the leptin promoter by 3-6 fold (n=2 each)(FIG. 12A). Blockade of glucose metabolism with 2-deoxy-D-glucose (2-DG)(10 mg/dl) markedly inhibited basal promoter activity and insulin-stimulated (10 nM) activation of the promoter (FIG. 12A). In the same experiments, the activity of the control plasmid (pGL2) was unaffected by either insulin (10 nM) or 2-DG (10 mg/dl)(FIG. 12B). These data provide evidence that the increase of leptin promoter activity induced by insulin is mediated via increased glucose metabolism (see Moreno-Aliaga, Biophys. Biochem, Res. Comm., 2001). Very similar results to these in 3T3-L1 cells have been found in new experiments in primary adipocytes (data not shown).

FIG. 13 includes graphs 13A and 13B which show the effects of DTNB (50 μM) and DCA (2 mM) on Absolute and Proportional Oxidation of Radiolabeled 14C-Glucose to CO2 in Isolated Rat Adipocytes: DTNB (n=8)(FIG. 13A) and DCA (FIG. 13B)(n=7) increased both the absolute rate of glucose oxidation and the proportion of glucose utilized that underwent oxidative metabolism (Data are expressed as percent of control; *=p<0.05 vs control).

FIG. 14A is a bar graph showing that the proportion of glucose metabolized to lactate decreased by 35% by the use of an antisense targeting PDHK induced shift from anaerobic to aerobic glucose metabolism.

FIG. 14B is a bar graph showing that the use of antisense targeting PDH-K increased leptin secretion by approximately 80%.

FIG. 14C is a line graph which shows that the decrease in anaerobic metabolism induced by antisense inactivation of PDHK is highly predictive of increased leptin secretion.

FIG. 15A is a bar graph showing the effect on β-Galactosidase activity in different cell cultures caused by an engineered adenovirus carrying the gene for β-Galactosidase.

FIG. 15B is a bar graph which shows that transfecting cells with an adenovirus containing the malic enzyme (ME) gene increased leptin secretion by 40% as compared to cells transfected with the β-Galactosidase virus.

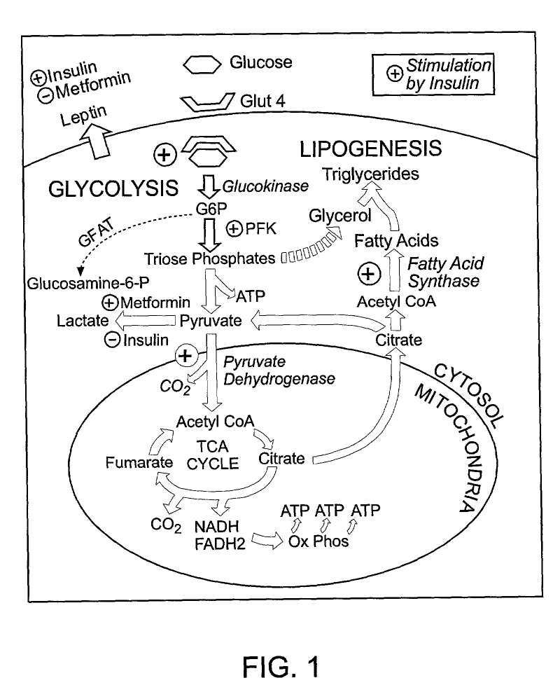

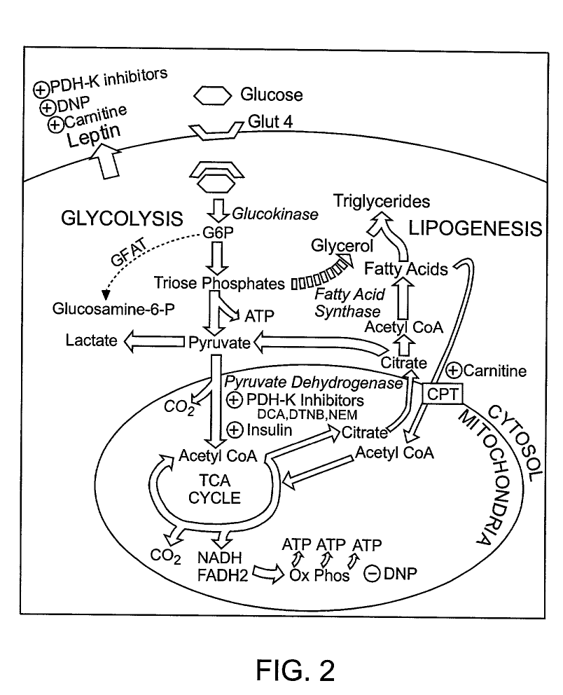

### DETAILED DESCRIPTION OF PREFERRED EMBODIMENTS

Before the present formulations and methods are described, it is to be understood that this invention is not limited to particular formulations and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “an enzyme” includes a plurality of such enzymes and reference to “the method” includes reference to one or more methods or steps and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present inventors have tested compounds which inhibit the activity of PDHK in an adipocyte culture system. At least three of these agents, 5,5′-Dithiobis(2-nitrobenzoate)(DTNB), Dichloroacetate (DCA), and N-ethylmaleimide (NEM) increase adipocyte glucose utilization, and/or decrease the anaerobic metabolism of glucose to lactate, and increase leptin production. These tests have shown that both DTNB and DCA increase glucose oxidation as assessed by the incorporation of radiolabeled glucose into CO₂. In addition, molecular inactivation of PDH kinase can be carried out by transfecting cultured adipocytes with antisense targeting PDH kinase decreases anaerobic glucose utilazation and increases leptin production. Accordingly, compounds with similar mechanisms of action to inhibit PDH kinase, or that act to increase PDH phosphatase, will augment glucose metabolism in adipose tissue and increase leptin production in vivo. Such compounds are useful for treating obesity and other conditions in which increased leptin production would have beneficial effects. Since the decrease of leptin is likely to contribute to increased hunger and decreased metabolic rate during energy-restricted diets, agents that increase endogenous leptin production are useful as an adjunct to diet and/or exercise to promote weight loss and to help prevent weight regain after successful dieting.

This invention describes the concepts underlying the use of agents that promote oxidative metabolism in adipose tissue as a method for stimulating leptin production for obesity treatment. In addition, the use of inhibitors of the enzyme PDH kinase, or antisense inactivation of PDH kinase, increases substrate metabolism (e.g., oxidation) and increase leptin production. These results show that this approach (metabolic activation) can be used to increase leptin production and also show that the use of specific compounds and formulations taught here are effective in stimulating leptin production in vitro. The present inventors have also shown that other mechanisms related to metabolism in adipose tissue including, but not limited to, NADPH malic enzyme, lactate dehydrogenase, fatty acid oxidation, and/or cellular ATP (adenylate charge) and redox status (NADH/NAD and NADPH/NADP ratios) are involved in the metabolic regulation of leptin production. Knowledge of such provides targets for the development of pharmacological methods to increase leptin production.

### Scope, Costs, and Complications of Obesity

Obesity is a serious, costly, and growing medical problem in the United States and throughout much of the world. Using the most stringent criteria, more than half of U.S. men and women age 20 and older are considered overweight (a body mass index (BMI)≧25 kg/m²), and nearly one-fourth are clinically obese (BMI≧30 kg/m²) (Wickelgren, 1998, Hill, 1998). The economic costs of obesity and its related co-morbidities of Type-2 diabetes and cardiovascular complications (hyperlipidemia, hypertension, and heart disease) are enormous; close to $100 billion (Wolf, 1998).

Since the prevalence of obesity is increasing, obesity-related diseases will demand a growing portion of the nation's health-care resources in the next century unless this troublesome trend can be reversed. Treatment or prevention of obesity is likely to reverse or prevent the onset of Type-2 diabetes and other obesity-related diseases. However, since current medical treatments of obesity are largely ineffective, new approaches to obesity management are clearly needed.

Although significant weight loss can often be achieved through the implementation of energy-restricted diets and/or exercise, the success rate in maintain the weight loss is very low. Therefore, new therapies for obesity management are clearly needed. Adipose tissue metabolism and the adipocyte hormone, leptin, have a central role in the regulation of fuel metabolism and energy balance. Accordingly, a better understanding of the mechanisms involved in the regulation of adipocyte metabolism and leptin production may lead to new approaches for controlling obesity. The present invention provides pharmacological agents which augment leptin production and prevent the decrease of leptin during dieting and therefore attenuate the increase of appetite (hunger) and decline in energy expenditure (i.e., activity and metabolic rate) associated with restriction of energy intake.

### Leptin: Importance in Human Energy Balance

The adipocyte hormone leptin (Zhang et al, 1995) is involved in the regulation of body weight via its central actions on energy intake and expenditure (Caro et al, 1996). Evidence of a role for leptin as a hormonal signal from peripheral adipose stores to the central nervous system has primarily been based on rodent studies. However, more recent evidence, including reports that leptin deficiency (Montague et al, 1997), or defects in the leptin receptor (Clement et al, 1998), cause increased appetite leading to overeating and extreme obesity in humans demonstrates that leptin is a critical regulator of energy balance in humans as well as rodents (See Review, Havel, Am. J. Clin. Nutr., 1998, Am. J. Clin. Nutr., 1999, Proc. Nutr. Soc., 2000, Exp. Bio. Med. 2001, Curr. Opin. Lipidol., 2002).

### Leptin Responses to Energy Restriction

Circulating leptin concentrations decrease during energy restriction in humans and the decrease is much larger than would be expected for the smaller changes in body fat content (Dubuc, 1998). A decrease of leptin is linked to increased appetite during an energy-restricted diet in human subjects (Keim, 1998). Hyperphagia, in insulin-deficient diabetic rats is mediated by a decrease of circulating leptin (Sindelar, 1999). Furthermore, the fall of resting energy expenditure in response to fasting in rodents is prevented by leptin administration (Doring, 1998). Thus, decreased leptin production increases appetite and food drive, and contributes to the lowering of metabolic rate that is observed in humans during an energy-restricted diet. Since weight maintenance at a lower level of body adiposity is more difficult than achieving initial weight loss, a treatment which prevents the fall in leptin that accompanies energy-restricted diets will be beneficial in sustaining weight loss after successful dieting. Increasing leptin production during the period of dynamic weight loss will also increase the effectiveness of diet/exercise regimens in initiating weight loss.

### Peripheral Actions of Leptin

Leptin has a number of effects other than its central actions to reduce food intake and increase energy expenditure. There are leptin receptors in many peripheral tissues (see review, Tartaglia, 1997), including the liver, kidney, adipose tissue, ovary, and gastrointestinal tract. Leptin appears to have peripheral actions on fuel metabolism and substrate flux (Rossetti et al, 1997, Barzilai et al, 1997). That these actions may have profound long-term effects is suggested by studies showing that two weeks of hyperleptinemia after leptin gene transfection (Chen et al, 1997) or during leptin infusion from osmotic minipumps (Barzilai et al, 1997) led to a marked loss of body fat in rats, whereas pair-fed animals exhibited much more modest reductions of body fat.

### Other Important Biological Actions of Leptin

Leptin is also involved in regulating reproductive function (see Review, Cunningham et al, 1999) since ob/ob mice lacking leptin are infertile, but fertility is restored by leptin treatment (Chehab et al, 1996). Obese human patients with leptin deficiency exhibit hypogonadism (Strobel et al, 1998). Furthermore, leptin administration has been shown to accelerate the onset of puberty in rodents (Barash et al, 1996, Cheung et al, 1997, Chehab et al, Science, 1997). It has been proposed that leptin acts as a general signal of low energy status to the neuroendocrine axes; leptin administration reverses the changes of thyrotropin, ACTH, and gonadotropins in response to fasting in mice (Ahima et al, 1996) and energy-restricted rats (Kras et al, 2000).

Humans with leptin receptor defects are not only obese, but have impaired growth hormone and thyrotropin secretion (Clement et al, 1998). Low leptin levels, resulting from very low amounts body fat and decreased food intake, contribute to amenorrhea in women athletes (Laughlin et al, 1997) or anorexic patients (Kopp et al, 1997). Leptin has additional centrally- and peripherally-mediated effects on carbohydrate and lipid metabolism. Leptin administration has been shown to decrease glucose and hemoglobin A1c levels, and reduce plasma triglycerides in humans with low leptin levels, hyperlipidemia, and insulin-resistant diabetes resulting from lipodystophy (Oral, New Engl. J., Med., 2002). Therefore, increasing endogenous leptin production would be useful in the treatment of some forms of hyperlipidemia and diabetes. Other potential functions of leptin include direct inhibitory effects on insulin secretion (Kieffer et al, 1997, Emilsson et al, 1997, Ahren & Havel, 1999), actions on adrenal function (Bornstein et al, 1997, Cao et al, 1997), angiogenesis (Bouloumie et al, 1998, Sierra-Honigmann et al, 1998), hematopoiesis (Gainsford et al, 1996), pulmonary function (O'donnell et al, 1999) and immune function (Lofreda et al, 1998, Lord et al, 1998). Therefore, a pharmacological method which increase leptins production will provide therapeutic value in treating a number of conditions such as infertility or impaired function of the hypothalamic-pituitary neuroendocrine axes, including gonandotrophic, thyrotrophic and adrenocorticotrophic function.

In addition, to its potential utility in weight loss and weight loss maintenance in obesity, increasing endogenous leptin production through modulation of adipocyte metabolism provides a useful treatment for promoting immune function, angiogenesis and wound healing, and hematopoiesis.

### Regulation of Leptin Production In Vivo

Circulating leptin concentrations are correlated with adiposity in humans and animals (Maffei, et al, 1995, Ahren et al, 1997, Havel et al, 1996). However, adiposity is not the sole determinant of circulating leptin concentrations since plasma leptin decreases after fasting (Ahren et al, 1997, Weigle et al, 1997) and increases after refeeding (Weigle et al, 1997) with only minor changes of body adiposity. In humans, a diurnal pattern of leptin secretion has been described with the highest concentrations occurring between midnight and 2:00 A.M (Sinha et al, 1996). This nocturnal peak is related to insulin responses to meals (Laughlin and Yen, 1997, Saad et al, 1998), is entrained by meal timing (Schoeller et al, 1997), and does not occur if the subjects are fasted (Boden et al, 1996).

A weight-maintaining low fat/high carbohydrate diet increases energy expenditure in women (Havel et al, 1996). Furthermore, feeding a low fat/high carbohydrate diet results in significant weight loss, even when it is consumed ad libitum (Havel et al, 1996). Increases of circulating leptin and insulin in response to high carbohydrate feeding appear to lower the regulated level of adiposity by producing small but prolonged increases of metabolic rate, an effect that is likely to be mediated by increases of insulin and leptin.

Meals high in carbohydrate content result in higher leptin concentrations over a 24 hr period than high fat meals. This is shown in a study measuring leptin over 24 h in 19 normal weight women consuming either high fat/low carbohydrate meals (60%/20%) or low fat/high carbohydrate (20%/60%) meals (Havel et al, 1999). Meal-associated plasma insulin and glucose excursions were larger after low fat/high carbohydrate meals. Plasma leptin concentrations were higher 4-6 hr after breakfast and lunch and the nocturnal rise was augmented after low fat/high carbohydrate meals compared with high fat/low carbohydrate meals. Adipocyte glucose metabolism regulates leptin expression and secretion, increases of dietary fat content reduce leptin production via a mechanism that is likely to be related to decreased insulin-mediated glucose metabolism in adipose tissue. This reduction of leptin levels contributes to the effects of high fat diets to promote increased energy intake, weight gain, and obesity in animals (Ahren et al, 1997, Hill et al, 1992, Surwit et al, 1997) and humans (Horton et al, 1995, Tataranni et al, 1997) and the effect of low fat/high carbohydrate diets to promote weight loss (Havel et al, 1996). The effects of dietary carbohydrate to stimulate leptin production can be augmented by administering a pharmacological agent acting at the level of the adipocyte. Thus, administration of such an agent makes it possible to promote and maintain weight loss induced by low fat or energy-restricted diets by lowering the regulated level of body adiposity.

### Evidence for a Role of Insulin-Mediated Glucose Metabolism in Regulating Leptin Production

Glucose is an important regulator of leptin expression and secretion. This is demonstrated by showing that increases of leptin (ob) MRNA after glucose administration in mice are well correlated with plasma glucose concentrations (Mizuno et al, 1996). Such is further demonstrated by showing that the infusion of small amounts of glucose to prevent the decline of glycemia during fasting in humans also prevents the decrease of plasma leptin (Boden et al, 1996). Further the decrease of plasma leptin during marked caloric restriction in humans is better correlated with the decrease of plasma glucose than with changes of insulinemia (Dubuc et al, 1998). Still further low plasma leptin levels are acutely increased by insulin administration in proportion to the degree of glucose lowering in insulin-deficient diabetic rats (Havel et al, 1998) or in insulin-dependent diabetic human subjects (Havel et al, 1997). Lastly, high carbohydrate meals, which produce larger insulin and glycemic excursions, increase 24 hr plasma leptin concentrations in human subjects when compared with equicaloric high fat meals (Havel et al, 1999). Thus, insulin is a physiological regulator of leptin production. However, in experiments with insulin infusion, it is necessary to infuse significant amounts of glucose along with insulin to prevent hypoglycemia. Therefore, it was previously unclear whether the increased leptin production during insulin and glucose administration is due to a direct effect of insulin per se, or might be mediated indirectly via insulin's actions to increase glucose uptake and metabolism in adipose tissue.

### Effects of Insulin-Mediated Glucose Transport and Glycolysis

A number of early in vitro studies conducted in isolated adipocytes found that insulin stimulated leptin expression and secretion (Mueller, 1998; Russell, 1998). The present invention shows that glucose metabolism has a role in mediating insulin-induced leptin expression and secretion as opposed to a direct role on the insulin signal transduction pathway. Among the numerous actions of insulin to stimulate glucose utilization are the effects of insulin to stimulate glucose transport into cells by increasing the translocation of glucose transporters (GLUT4) to the plasma membrane. In addition insulin increases the flux of glucose through glycolysis primarily at the level phosphofructokinase (PFK) by increasing enzymatic production of fructose-2,6 bisphosphate, an allosteric activator of PFK (Tepperman, 1980)(Schematic Diagram 1).

### Adipocyte Culture System

To investigate the role of adipocyte metabolism in regulating leptin production, the present invention provides a culture system in which freshly isolated mature rat adipocytes are maintained in culture anchored to a basement membrane matrix (Matrigel) or collagen. Although all in vitro systems have inherent advantages and disadvantages, advantages of this system compared with cultures containing free-floating adipocytes are (1) that the matrix simulates their normal basement membrane attachment and (2) that the cells are maintained in close proximity to each other, allowing direct cell-to-cell contact. Together the cell contact and basement membrane attachment help to maintain differentiation, since adipocytes have a strong tendency to dedifferentiate in long-term (>24 h) culture. Furthermore, the adipocytes in this system are not exposed to toxic levels of oxygen at the interface of the media and the incubator atmosphere, as opposed to free-floating adipocytes, which aggregate at the surface of the media. An advantage of the system over those containing minced adipose tissue is that all of the cells in the culture are equally exposed to the nutrients and the oxygen dissolved in the media. Thus, although clearly different from the in vivo situation, we believe that this system provides a more physiological environment than most systems for maintaining adipocytes in long-term culture.

The present invention demonstrates that glucose utilization by adipocytes is required for insulin-stimulated leptin expression and secretion. The results obtained show that leptin secretion is proportional to the rate of glucose utilization. Other experiments demonstrate that leptin secretion and ob gene expression are suppressed when glucose transport and phosphorylation are inhibited with 2-deoxy-D-glucose (2-DG) treatment. Furthermore, leptin expression and secretion are reduced when glycolysis was suppressed with sodium fluoride (Mueller, 1998). Other inhibitors of glucose transport and utilization had similar effects to inhibit leptin production. The suppression of leptin production by all of the agents examined was proportional to actions of the compounds to inhibit adipocyte glucose utilization.

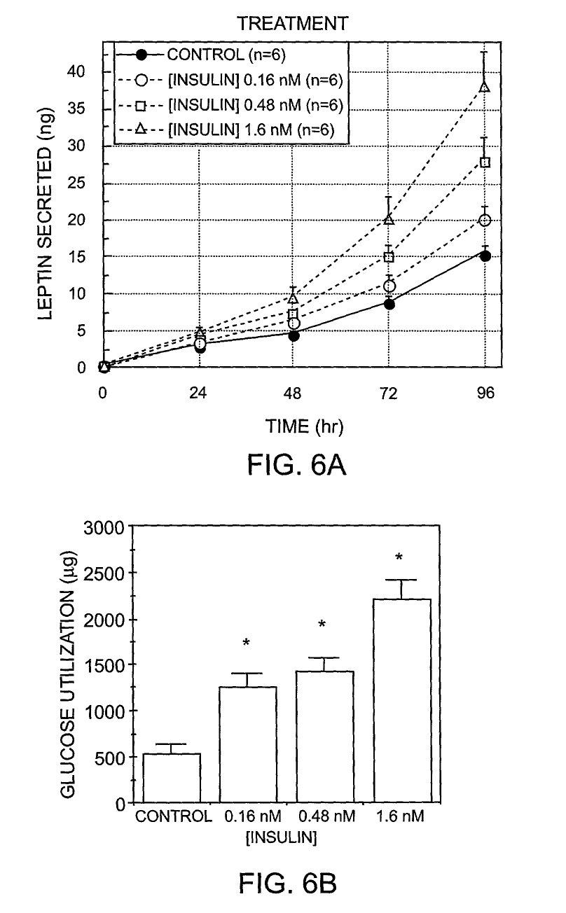

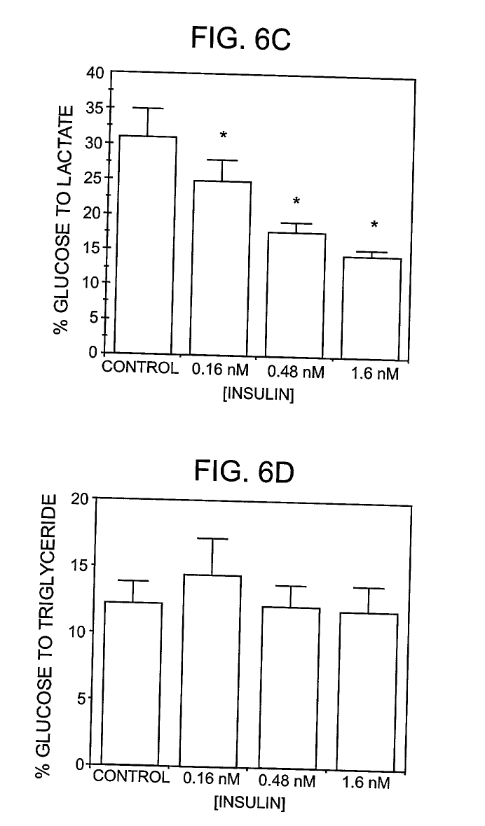

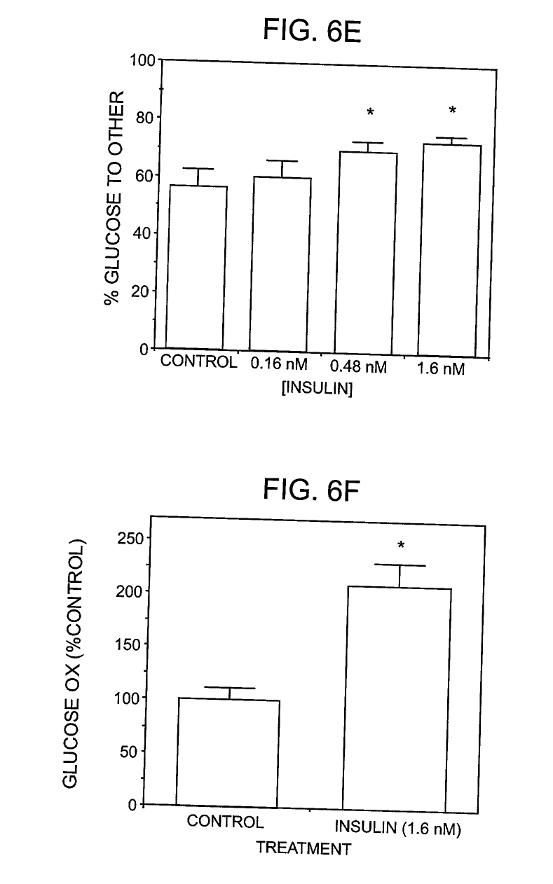

FIGS. 6A-6D illustrate the effects of increasing concentrations of insulin within a physiological range (0.16-1.6 nM) on adipocyte metabolism and leptin production. As outlined above, insulin induces a concentration dependent increase in leptin secretion (FIG. 6A) and glucose utilization (FIG. 6B).

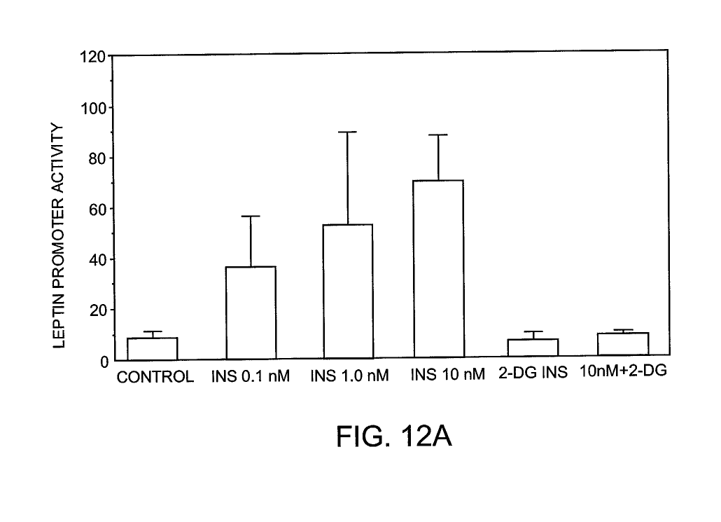

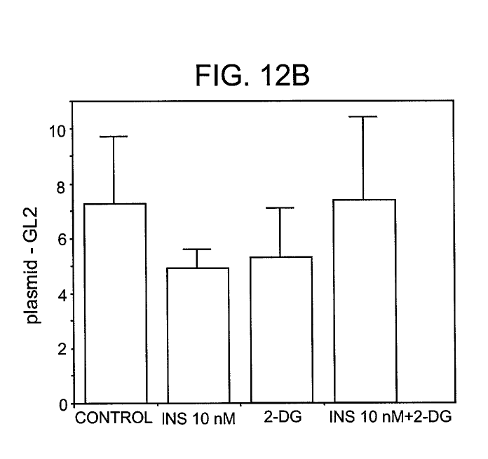

Insulin (0.1 to 10 nM) also increases the transcriptional activity of a luciferase construct driven by the leptin promoter when it is transfected into 3T3-L1 adipocytes, an effect that is completely blocked when glucose metabolism is inhibited with 2-DG (FIG. 12A). In contrast the activity of a control plasmid expressing β-galactosidase was unaffected by insulin or 2-DG (FIG. 12B), suggesting that insulin and 2-DG are not exerting generalized effects on cellular transcriptional activity (See Moreno-Aliaga et al, Biochem. Biophy. Res. Comm., 2001). These results from experiments in 3T3-L1 cells have now been replicated in primary adipocytes in which the activity of a transfected construct of the leptin promoter is increased by insulin and the effect of insulin is blocked by inhibiting glucose metabolism with 2-DG. In contrast the activity of the a control plasmid was unaffected by insulin or 2-DG.

### Role of Aerobic Metabolism

Insulin: 1) decreases the proportion of glucose that is anaerobically metabolized to lactate (FIG. 6C), 2) does not alter the proportion of glucose that is incorporated into triglyceride (FIG. 6D), and 3) by subtraction increases the proportion of glucose that is not converted to lactate or triglyceride (FIG. 6E).

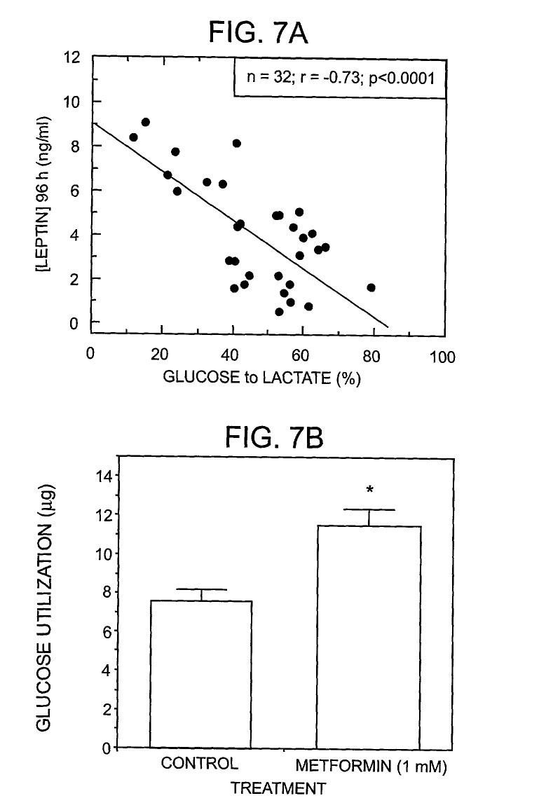

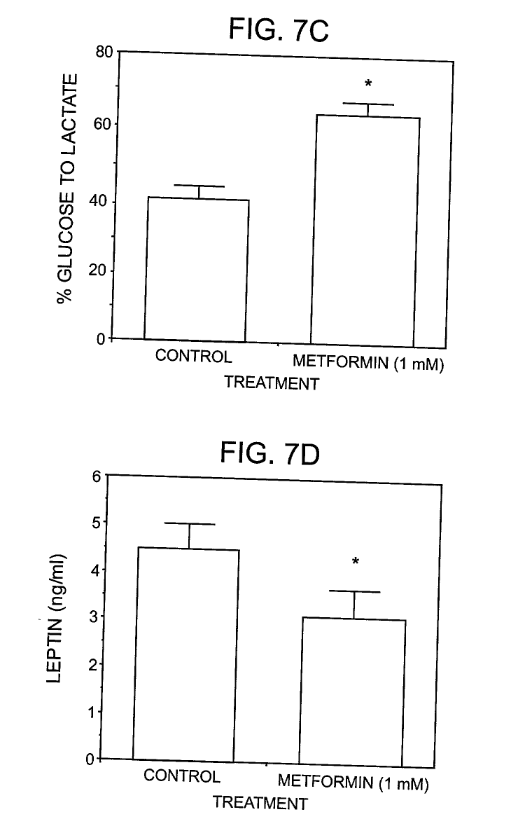

This glucose was subjected to mitochondrial oxidation and the present invention shows that insulin at a concentration of 1.6 nM markedly increases glucose oxidation as assessed by the incorporation of ¹⁴C-labeled glucose into CO₂ (FIG. 6F). The Examples show that glucose transport per se is not the regulatory step in leptin production by adipocytes. Rather, glucose transport and phosphorylation are necessary in order for glucose to be further metabolized. The Examples also show that leptin secretion is inversely related to the rate of conversion of glucose to lactate (FIG. 7A). This shows that anaerobic metabolism of glucose does not stimulate leptin production. Additional studies with metformin revealed that metformin inhibits leptin secretion by diverting glucose into an anaerobic pathway generating lactate (FIGS. 7B-D). Based on these results and other factors we have deduced that the metabolism of glucose beyond pyruvate, to a fate other than lactate, causes effects on glucose metabolism to stimulate leptin production. Further other pathways of glucose metabolism which are stimulated by insulin in adipose tissue particularly mitochondrial metabolism are involved in the regulation of leptin production by glucose.

### Role of Glucose Oxidation

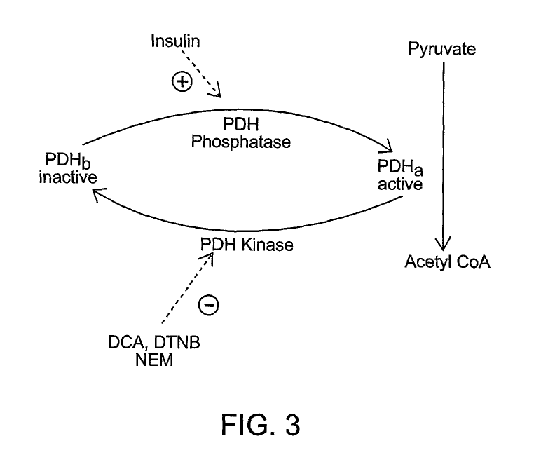

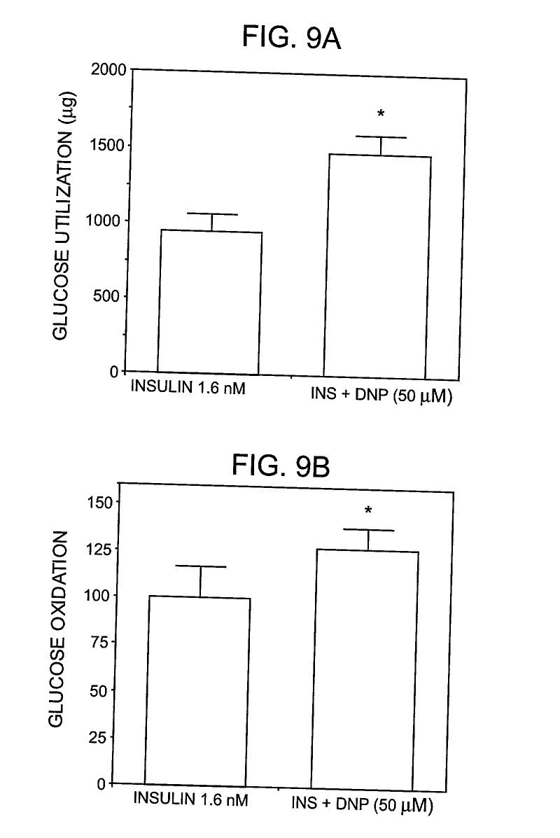

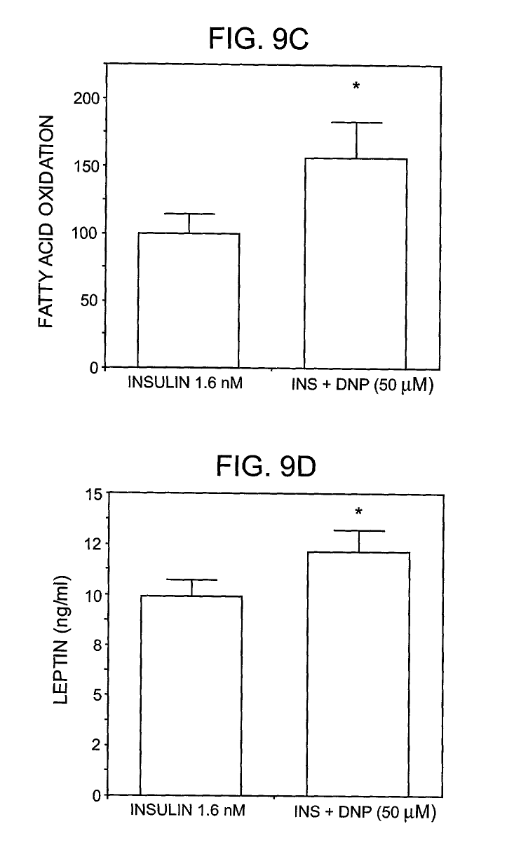

Other data shows the connection between oxidative metabolism and the regulation of leptin secretion. The uncoupling agent, dinitrophenol (DNP), at low concentrations, markedly increases glucose utilization (FIG. 9A), glucose and fatty acid oxidation (FIGS. 9B and 9C), and stimulates leptin secretion (FIG. 9D). The increase in glucose utilization is a compensatory response to reduced ability to generate ATP. Thus, under these conditions, the flux of substrate into and through the TCA cycle is increased. Another method for increasing the flux of carbon from glucose into the TCA cycle is to increase the activity of pyruvate dehydrogenase (PDH)(see FIG. 3 for an overview of PDH regulation).

The enzyme PDH kinase (PDHK) is a negative regulator of PDH activity. When PDH is phosphorylated by PDHK, its activity is decreased and less glucose carbon can enter the mitochondria for oxidation through the TCA cycle. Insulin increases PDH activity by activating a PDH phosphatase enzyme (Taylor, 1973), which dephosphorylates PDH (see FIG. 3). Therefore if PDHK is inhibited or if PDH phosphatase is activated, PDH activity will increase and more glucose can be oxidized. This would stimulate leptin production.

### Role of PDH and PDH-K

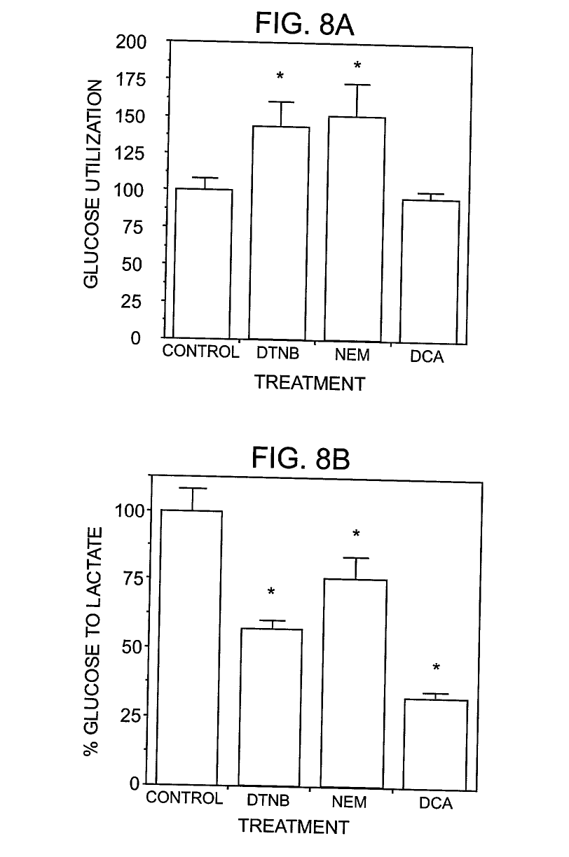

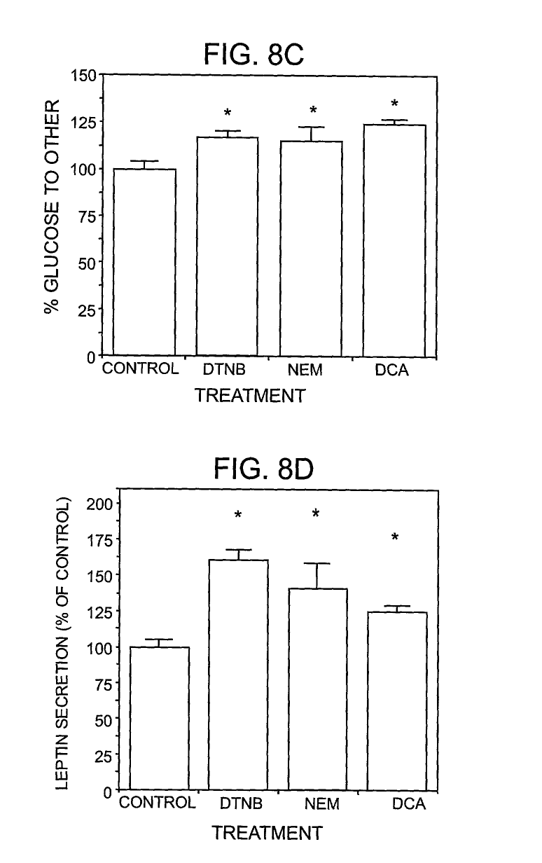

The results provided here show that specific inhibitors of PDHK increase PDH activity in adipocytes and stimulate leptin production. PDHK inhibitors are described in Proc. Natl. Acad. Sci. USA, 19:3945-3948 (1982). The inhibitors, N-ethylmaleimide (NEM) and 5,5′-Dithiobis(2-nitrobenzoate)(DTNB) were tested in the adipocyte culture system, FCC 136, Vol. 3, page 127). The analyses of the glucose and lactate results from that experiment showed that DTNB at a concentration of 100 μM and NEM at a concentration of 0.1 μM increased insulin-mediated glucose utilization (FIG. 8A) without any increase of lactate production. Specifically these two PDHK inhibitors decreased the proportion of glucose carbon released as lactate (FIG. 8B), without affecting the proportional incorporation of labeled glucose into triglyceride. By subtraction it was shown that these inhibitors increased the proportional glucose flux into oxidation (FIG. 8C).

The same concentrations of DTNB (100 μM) increased leptin production by approximately 60% and NEM (0.1 μM) increased leptin secretion by 25% (FIG. 8D).

Another compound reported to be a PDH kinase inhibitor, dichloroacetate (DCA), at a concentration of 2 mM, did not increase total glucose utilization (FIG. 8A), but markedly lowered anaerobic metabolism of glucose to lactate (FIG. 8B), did not increase the proportional incorporation of labeled glucose into triglycerides, and by subtraction increased glucose oxidation (FIG. 8C), and increased leptin secretion by 20-25% (FIG. 8D).

These results provide strong evidence that increasing glucose carbon flux into the mitochondria through PDH for oxidative metabolism by inhibiting PDHK is a viable approach for increasing leptin production by adipose tissue. The effects of DTNB, NEM, and DCA on glucose utilization, lactate production, the proportional anaerobic conversion of glucose to lactate, and leptin secretion are summarized in Tables 1-3, respectively. Therefore, PDHK inhibitors enhance leptin production and are useful in the management, including treatment of obesity and the prevention of weight regain after weight loss.

TABLE 1
Effects of 5,5′-Dithiobis(2-nitrobenzoate)(DTNB) in the presence of 0.48 nM insulin on glucose utilization, lactate
production, the percentage of glucose carbon taken up that was released as lactate, and leptin production by isolated rat adipocytes
over 96 hours in culture.

| DTNB (μM) + | Glucose Uptake | Lactate Produced | Glucose to | Leptin (ng) Produced | % Leptin Produced | [Leptin] | % [Leptin] |
|---|---|---|---|---|---|---|---|
| Insulin (0.48 nM) | (μg) over 96 h | (μg) over 96 h | Lactate (%) | over 96 h | over 96 h | (ng/ml) at 96 h | (ng/ml) at 96 h |
| Control | 1035 ± 86 | 231 ± 27 | 22.6 ± 1.8 | 37.1 ± 2.2 | — | 10.9 ± 0.6 | — |
| (n = 11) | | | | | | | |
| 100.0 (n = 11) | 1384 ± 94 | 179 ± 22 | 13.1 ± 1.4 | 55.6 ± 2.9 | — | 17.2 ± 0.9 | — |
| Change in | +348 ± 114 | −53 ± 25 | −9.5 ± 1.0 | +18.4 ± 2.3 | +57.3 ± 5.8 | +6.3 ± 0.8 | +60.2 ± 8.3 |
| Parameter | | | | | | | |
| t-value | 3.05 | 2.12 | 9.50 | 8.00 | 9.88 | 7.88 | 7.25 |
| p-Value | p < 0.01 | 0.05 | P < 0.0001 | P < 0.0001 | P < 0.0001 | P < 0.0001 | P < 0.0001 |

TABLE 2
Effects of N-ethylmaleimide (NEM) in the presence of 0.48 nM insulin on glucose utilization, lactate production, the
percentage of glucose carbon taken up that was released as lactate, and leptin production by isolated rat adipocytes over 96 hours in culture.

| NEM (μM) + | Glucose Uptake | Lactate Produced | Glucose to | Leptin (ng) Produced | % Leptin Produced | [Leptin] | % [Leptin] |
|---|---|---|---|---|---|---|---|
| Insulin (0.48 nM) | (μg) over 96 h | (μg) over 96 h | Lactate (%) | over 96 h | over 96 h | (ng/ml) at 96 h | (ng/ml) at 96 h |
| Control (n = 6) | 970 ± 80 | 188 ± 15 | 20.6 ± 2.0 | 49.2 ± 4.5 | — | 14.5 ± 1.3 | — |
| 0.1 (n = 6) | 1399 ± 132 | 178 ± 11 | 13.6 ± 1.5 | 63.4 ± 8.2 | — | 18.4 ± 2.4 | — |
| Change in | +348 ± 114 | −10 ± 13 | −6.9 ± 2.3 | +14.2 ± 4.2 | +26.6 ± 6.2 | +4.0 ± 1.0 | +25.3 ± 6.3 |
| Parameter | | | | | | | |
| t-value | 3.05 | 0.77 | 3.0 | 3.38 | 4.29 | 4.00 | 4.02 |
| p-Value | p < 0.02 | NS | P < 0.02 | P < 0.01 | P < 0.005 | P < 0.01 | P < 0.01 |

TABLE 3
Effects of dichloroacetate (DCA) in the presence of 0.48 nM insulin on glucose utilization, lactate production, the
percentage of glucose carbon taken up that was released as lactate, and leptin production by
isolated rat adipocytes over 96 hours in culture.

| DCA (mM) + | Glucose Uptake | Lactate Produced | Glucose to | Leptin (ng) Produced | % Leptin Produced | [Leptin] | % [Leptin] |
|---|---|---|---|---|---|---|---|
| Insulin (0.48 nM) | (μg) over 96 h | (μg) over 96 h | Lactate (%) | over 96 h | over 96 h | (ng/ml) at 96 h | (ng/ml) at 96 h |
| Control (n = 6) | 1129 ± 92 | 263 ± 9 | 24.1 ± 2.0 | 75.4 ± 6.3 | — | 22.3 ± 1.8 | — |
| 2.0 (n = 6) | 1082 ± 117 | 81 ± 4 | 7.9 ± 0.9 | 88.3 ± 7.5 | — | 27.0 ± 2.6 | — |
| Change in | −46 ± 57 | −181 ± 10 | −16.1 ± 1.3 | +12.9 ± 3.6 | +17.6 ± 4.7 | +3.6 ± 1.0 | +17.9 ± 7.9 |
| Parameter | | | | | | | |
| t-value | NS | 18.10 | 12.38 | 3.58 | 3.74 | 3.60 | 2.27 |
| p-Value | p < 0.01 | P < 0.0001 | P < 0.0001 | P < 0.01 | P < 0.01 | P < 0.01 | P < 0.05 |

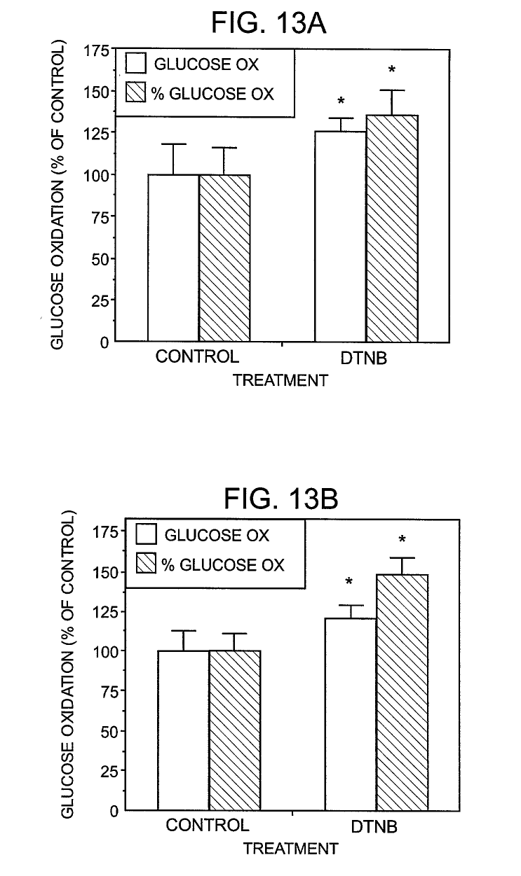

Additional experiments tested the effects of DTNB and DCA on glucose oxidation as assessed by the incorporation of radiolabeled glucose carbon into CO₂. In these experiments, DTNB and DCA increased glucose oxidation by isolated adipocytes (FIGS. 13A and 13B). In addition, to its effects on diverting glucose away from anaerobic metabolism to lactate, DCA also decreases the concentration of lactate present at the start of the incubations, showing that DCA promotes the conversion of lactate to pyruvate. This is a result of increased lactate to pyruvate flux through an isoform of lactate dehydrogenase that coverts lactate to pyruvate. Therefore, a method to increase lactate metabolism to pyruvate would also enhance leptin production.

### Role of Malic Enzyme and NADPH

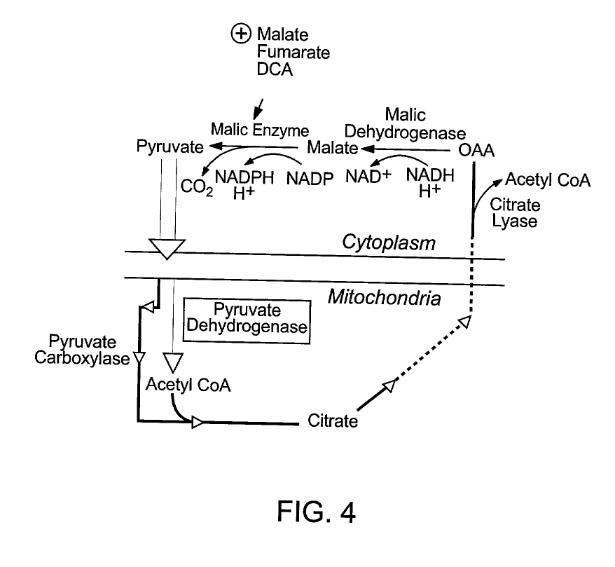

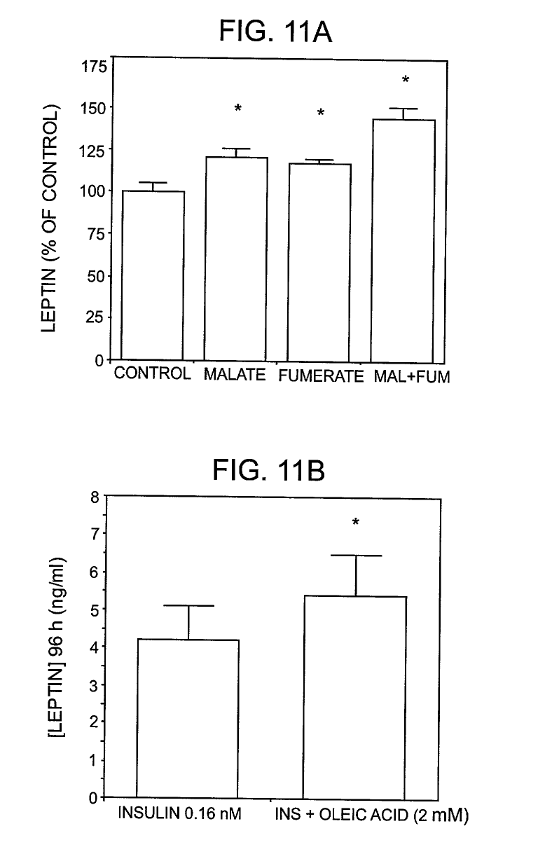

In the fed state (i.e. high insulin and increased glucose flux), the pyruvate-malate cycle serves to transport acetyl-CoA from the mitochondria to the cytosol and to generate NADPH via the action of malic enzyme (see FIG. 4). Acetyl-CoA units are transported from the mitochondria in the form of citrate via a tricarboxylic acid carrier. Citrate stimulates leptin secretion in the presence of low glucose and insulin concentration (Rudolph et al, 1997), whereas in a situation when citrate flux out of the mitochondria is already increased (presence of high insulin and glucose), citrate does not affect leptin secretion. Therefore, citrate could either enter the mitochondria for oxidation in the TCA cycle, or be cleaved by citrate lyase with the OAA generated being converted to malate (via malate dehydrogenase) and then to pyruvate via malic enzyme. That the flux of substrate through malic enzyme may be important in regulating leptin production is suggested by the results of several experiments. First, in addition to inhibiting PDHK, DCA is known to stimulate malic enzyme activity (Mann, 1992) and this action might be involved in its effect to increase leptin secretion (see above). Since concentrations of DCA from 0.1 to 5.0 mM all markedly lowered lactate production to a similar extent, but 2.0 mM was the most potent in stimulating leptin secretion, DCA may increase leptin secretion by another mechanism in addition to inhibition of PDHK, and this could be by activating malic enzyme. Second, the addition of exogenous malate to the culture system of the present invention modestly stimulates leptin production (+20%) in the presence of low insulin and glucose (FIG. 11A). Third, fumarate, which is known to an allosteric activator of malic enzyme (Moreadith, 1984) also increases leptin secretion (+20%), and enhances the stimulation of leptin secretion by malate to approximately +50% over control (FIG. 11A), suggesting that increased flux into the malate-pyruvate cycle is a regulator of leptin production. Thus, the effects of citrate and malate to stimulate leptin in the presence of low glucose provide further support for a role for mitochondrial metabolism and the pyruvate-malate cycle in the effects of glucose metabolism to increase leptin production. An increase of NADPH by malic enzyme may be a cytosolic signal of increased energy flux into mitochondrial metabolism

### Role of Fat Oxidation

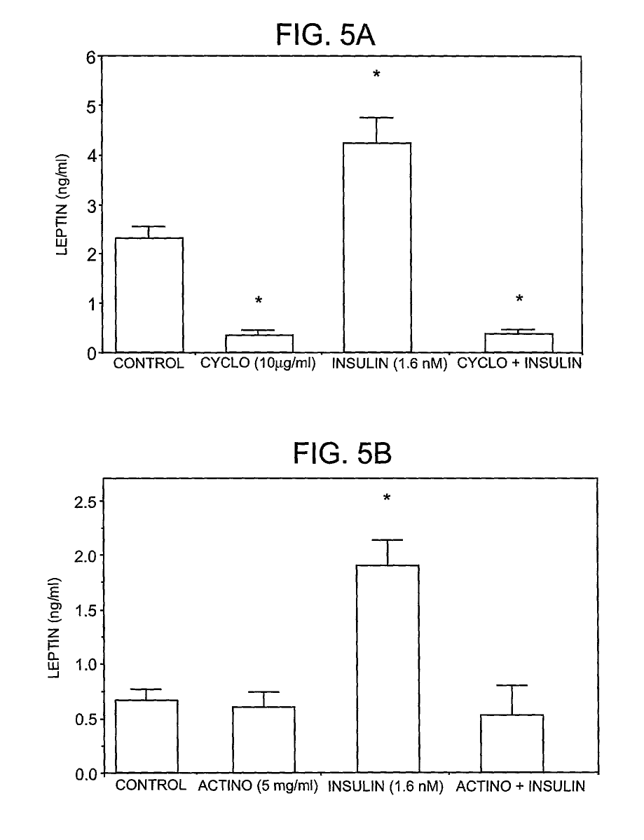

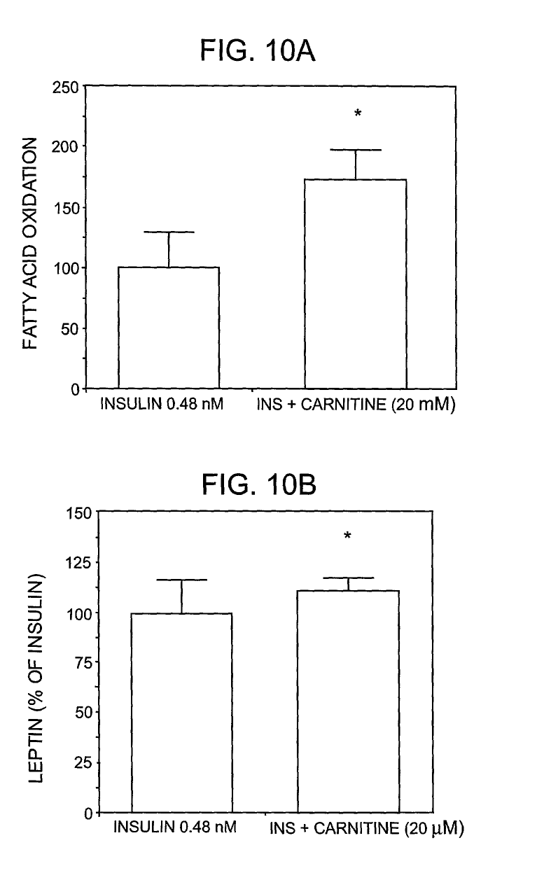

The results of three different experiments suggest that increases of fatty acid oxidation may stimulate leptin production. As discussed above a moderate concentration of the uncoupling agent, DNP, modestly increased glucose oxidation (FIG. 5B) and leptin secretion (FIG. 9D). Experiments were also carried out to examine fatty acid oxidation by measuring the incorporation of ¹⁴C-labeled oleate into CO₂. Thus, uncoupling with DNP increases both carbohydrate and lipid oxidation (FIGS. 9B and 9C), perhaps as a compensatory mechanism to produce energy from any available substrate when ATP production is suppressed. Therefore, to further examine the potential role of fatty acid oxidation adipocytes were incubated with L-camitine, a cofactor of the rate-limiting step for fatty acid transport into the mitochondria via camitine-palmitoyl-tranferase (CPT). Carnitine treatment increased fatty acid oxidation (FIG. 10A), inhibited glucose utilization, glucose oxidation, and glucose incorporation into lipid (data not shown), and modestly increased leptin secretion (FIG. 10B). These results provide additional evidence that lipid oxidation, in addition to glucose oxidation, can increase leptin production. Lastly, the addition of oleic acid (2 mM) in the presence of low glucose inhibits glucose oxidation and increases leptin secretion (FIG. 11B).

### Role of Energy and Redox Potential (ATP, NADH, and NADPH)

The redox potential of the adipocyte is another mechanism by which substrate metabolism could lead to increased leptin production. In glycolysis, NADH is formed at the glyceraldehyde 3-phosphate dehydrogenase (G-3-P-DH) step. If the pyruvate formed at the end of glycolysis is anaerobically metabolized to lactate, NADH is taken to NAD and there is no net increase of NADH or the NADH/NAD ratio. The formation of lactate allows glycolysis to continue under anaerobic conditions since NAD is reformed and the flux through G-3-P-DH can continue. Without the reformation of NAD, glycolysis would back up and no glucose would be utilized. If the pyruvate from glycolysis is metabolized via PDH and enters the mitochondria then NAD in the cytosol needs to be regenerated via the malate/aspartate or glycerol phosphate shuttle systems in order for glycolysis to continue.

The pyruvate-malate cycle plays a role in mediating insulin-induced leptin secretion. A key step in this cycle is the conversion of malate to pyruvate via malic enzyme (FIG. 4). Malate and its allosteric activator increase leptin secretion. Activation of malic enzyme could contribute to the effect of DCA to increase leptin secretion (FIG. 8D). Pyruvate in the absence of insulin and glucose stimulates leptin secretion. However, the present invention shows that in the presence of glucose and insulin pyruvate actually inhibits leptin secretion. Thus pyruvate may be exerting an end-product inhibition of malic enzyme and thereby reducing flux through the pyruvate-malate cycle. This is similar to the effects of citrate and malate to stimulate leptin secretion in the presence of low, but not higher, levels of insulin and glucose. The conversion of malate to pyruvate via malic enzyme generates NADPH. NADPH is an important contributor to the cellular redox state and in addition supplies reducing energy used in fatty acid synthesis. Although NADPH can also be produced via the pentose phosphate pathway, the production of NADPH from that pathway is coupled to fatty acid synthesis and NADPH is used as lipogenesis proceeds. In contrast the NADPH generated by malic enzyme is not necessarily used for lipogenic purposes and therefore may serve as a signal of cellular energy surplus, which is the condition under which leptin production is increased in adipose tissue.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### MATERIALS AND METHODS FOR ADIPOCYTE CULTURE

Materials: Media (DMEM) and fetal bovine serum (FBS) are purchased from Life Technologies (Grand Island, NY). The media is supplemented with 6 ml each of MEM nonessential amino acids, penicillin/ streptomycin (5000 U/ml/5000 ug/ ml), and nystatin (10,000 U/ml; all from Life Technologies) per 500 ml DMEM. Bovine serum albumin (BSA) fraction V, HEPES, collagenase (Clostridium histolyticum; type II, SA 456 U/mg), insulin, NEM, and DTNB are purchased from Sigma Chemical Co (St. Louis, Mo.). Matrigel matrix is purchased form Becton Dickinson (Franklin Lakes, N.J. Collagen is purchased from Cohesion Technologies, (Palo Alto, Calif.). Nylon filters are purchased from Tetko (Kansas City, Mo.).

Animals: Results were obtained using isolated rat adipocytes. However, techniques described here can be conducted in isolated mouse adipocytes. (Gregoire F, Stanhope K L, Havel P J, West D B. Functional assessment of insulin-stimulated glucose utilization in cultured adipocytes derived from C57BL/6J and DBA/2J inbred mice. Obesity Res. 8 (Suppl. 1): 66S, 2000). Male Sprague-Dawley rats (3-6 months of age) are obtained from Charles River (Wilmington, Mass.) or Harlan Sprague-Dawley. Animals are housed in hanging wire cages in temperature controlled rooms (22° C.) with a 12-h light-dark cycle and fed Purina chow diet (Ralston-Purina, St. Louise, Mo.) and given deionized water ad libitum. Animal use and care is in accordance with the National Institutes of Health Guide for the Use and Care of Laboratory Animals and conducted in facilities accredited by the American Association for Accreditation of Laboratory Animal Care (AAALAC). The study protocols have been approved to the Administrative Animal Use and Care Committee at the University of California, Davis.

### Methods

Cell isolation/ preparation: Adipocytes are prepared from epididymal fat pads from male Sprague-Dawley rats weighing 300-600 g. Epididymal fat depots are resected from halothane anesthetized rats under aseptic conditions and adipocytes are isolated by collagenase digestion by the Rodbell method (Rodbell M. Metabolism of isolated fat cells. I. Effects of hormones on glucose metabolism and lipolysis. J Biol Chem. 1964;239: 375-380), with minor modifications as previously described (Mueller W M, Gregoire F M, Stanhope K L, Mobbs C V, Mizuno T M, Warden C H, Stern J S, Havel P J. Evidence that glucose metabolism regulates leptin secretion from isolated adipocytes. Endocrinology 139: 551-558, 1998; Mueller W M, Stanhope K L, Gregoire F, Evans J L, Havel P J. Effects of metformin and vanadium on leptin secretion from cultured rat adipocytes. Obesity Res. 8: 530-539, 2000; Medina E A, Stanhope K L, Mizuno T M, Mobbs C V, Gregoire F, Hubbard N E, Erickson K L, Havel P J. Effects of tumor necrosis factor alpha on leptin secretion and gene expression: relationship to changes of glucose metabolism in isolated rat adipocytes. Int J Obes Relat Metab Disord. 23: 896-903, 1999.). The isolated adipocytes are then incubated for 30 minutes at 37C. before being plated and cultured on Matrigel-coated plates.

Adipocyte Culture: Adipocytes are maintained in culture anchored to a basement membrane matrix (Matrigel, Becton Dickinson, Franklin Lakes, N.J.) or collagen from Cohesion Technologies, (Palo Alto, Calif.). Although all in vitro systems have inherent advantages and disadvantages, advantages of this system compared with cultures containing free-floating adipocytes are that the matrix simulates their normal basement membrane attachment and that the cells are maintained in close proximity to each other, allowing direct cell to cell contact. Together the cell contact and basement membrane attachment help to maintain differentiation, since adipocytes have a strong tendency to dedifferentiate in long-term (>24 h) culture. In addition, the matrix and the small amount of serum in the media both contain growth factors, which are also likely to help in maintaining cell differentiation. Furthermore, the adipocytes in this system are not exposed to toxic levels of oxygen at the interface of the media and the incubator atmosphere, as opposed to free-floating adipocytes which aggregate at the surface of the media. An advantage of the system over those containing minced adipose tissue is that all of the cells in the culture are equally exposed to the nutrients and the oxygen dissolved in the media. Thus, although clearly different from the in vivo situation, this system provides a more physiological environment than most systems for maintaining adipocytes in long-term culture.

The goal of these experiments was to examine the direct actions of metformin and vanadium on leptin production and adipocyte metabolism. Therefore, the advantage of employing in vitro experimentation for this purpose over in vivo models is that it was possible to control confounding variables, such as effects of these agents on food intake (Havel P J. Mechanisms regulating leptin production: implications for control of to energy balance. Am J Clin Nutr. 1999;70:305-306;. Havel P J. Role of adipose tissue in body-weight regulation: mechanisms regulating leptin production and energy balance Proc. Nutr. Soc. 59: 359-371, 2000), which would indirectly influence leptin production via changes of insulin secretion (Saad M F, Khan A, Sharma A, et al. Physiological insulinemia acutely modulates plasma leptin. Diabetes. 1998; 47: 544-549; Havel P J, Townsend R, Chaump L, Teff K. High fat meals reduce 24 hour circulating leptin concentrations in women, Diabetes. 1999;48:334-341). Unlike an in vivo system, in these experiments the environment surrounding the adipocytes within the individual wells of each culture plate was identical with the exception of the presence or absence and the concentration of metformin or vanadium, allowing assessment of the direct effects of the treatments.

In culturing each suspension, Matrigel is first thawed on ice to a liquid and uniformly applied to the surface of culture dishes (300 μl Matrigel/35 mm well). After the incubation period, 150 μl of the adipocyte suspension (2:1 ratio of packed cells to media) are plated on the Matrigel or collagen martix. Adipocytes from each suspension are thoroughly mixed with a transfer pipette before plating to insure that a similar number adipocytes with a similar size distribution are added to the control and experimental wells for each suspension. The warmth of the added cells and buffer causes the Matrigel to gel around the adipocytes, or the neutralization of the acidic pH of the collagen solution to ˜7.0 solidifies the collgen, and both of these techniques effectively anchor the adipocytes to the culture dish. After a 30 minute incubation at 37° C., 2 ml of warm culture medium is added. The cells are maintained in an incubator at 37° C. for 96 hours with 6% CO₂. Aliquots of adipocytes from each animal are divided into wells, with the different concentrations of insulin or other agents to be tested. In each plate an appropriate control well contains adipocytes from the same animal. Adipocytes are incubated with media (DMEM) containing 5.5 mM (100 mg/dl) glucose plus 5% FBS at several concentrations of inhibitors to be tested. In all experiments, aliquots of media, 300 μl, (15% of the media volume) is collected from culture wells and replaced with fresh media containing the appropriate concentrations of insulin or other agents to be tested at 24, 48, 72, and 96 hours.

Incorporation of Glucose Carbon into Triglyceride: To measure glucose incorporation into triglyceride, cultures are exposed to media containing 0.01 uCi/ml of ¹⁴C-glucose. After 96 hr, media and extracellular lipid is removed from the well and methanol added. Then scrape the collagen-cell matrix from the CO₂ of the total DPM remaining in the media at 48 hours and as micromoles of substrate oxidized over time.

Northern Blot Procedure: RNA is extracted according to the Gibco Life Technologies procedure using Trizol (Life Technologies Inc., Grand Island, N.Y.). UV absorbance and integrity gels is used to estimate RNA. The cDNA probe for leptin has been kindly provided by Dr. Charles Mobbs (Mount Sinai School of Medicine, New York). The cDNA probes for malic enzyme; CPT and PDH are purchased from Molecular Probes, Eugene, Oreg. cDNA probes are labeled by random priming (Rediprime kit, Amersham) in the presence of ³²P dCTP (3000 Ci/mmol, Amersham). Unincorporated nucleotides are removed using NucTrap probe purification columns (Stratagene, La Jolla, Calif.). For each tissue sample, 5-10 μg of total RNA is fractionated by electrophoresis on a denaturing 1% agarose gel containing 2.2 M formaldehyde and 1× MOPS running buffer. One μl of a 50 μg/ml ethidium bromide stock solution is added in order to check RNA integrity and even loading. After electrophoresis, RNA is transferred onto a nylon membrane (Duralon-UV, Stratagene, La Jolla, Calif.) by overnight capillary transfer and UV cross-linked (Stratalinker 1800, Stratagene, La Jolla, Calif.). Blots are hybridized for 1 hr at 68° C. in presence of the labeled cDNA probe (2×10⁶ cpm/ml Express hyb solution). Blots are washed 2× at high stringency and exposed to X-ray films with an intensifying screen for 2 days at −80° C. (Kodak BioMax). Leptin mRNA is analyzed using a single-stranded cDNA probe and quantified using a phosphoimager. Blots are analyzed again using a probe complementary to mouse 18S ribosomal RNA. mRNA levels are normalized with respect to the 18S ribosomal RNA signal.

Assays: Leptin concentrations in the medium are determined with a sensitive and specific RIA for rat leptin (Landt M, Gingerich R L, Havel P J, Mueller W M, Schoner B, Hale J E, Heiman M L. Radioimmunoassay of rat leptin: Sexual dimorphism reversed from humans. Clin Chem. 1998;44:565-570) or for mouse leptin (Ahren B, Mansson S, Gingerich R L, Havel P J. Regulation of plasma leptin in mice: influence of age, high-fat diet, and fasting. Am. J. Physiol. 273: R113-120, 1997) with reagents obtained from Linco Research, St. Charles, Mo. Glucose and lactate are measured with a YSI glucose analyzer (Model 2300, Yellow Springs Ins., Yellow Springs, Ohio).

Data Analysis: The uptake of glucose is assessed by measuring the concentration of glucose in the media in each well before and at 24, 48, 72, and 96 hours of incubation and calculating the decrease over 96 hours, after correcting for the amount of glucose that was removed during each 24 h media sampling and the amount added by the replacement of fresh media (15% of total volume). Lactate production is calculated as the increase of media lactate at 24, 48, 72, and 96 hours, correcting for the amount of lactate removed by sampling and added with media replacement. To examine the relationship between adipocyte carbon flux and leptin secretion in adipocytes, the amount of carbon released as lactate per amount of carbon taken up as glucose over 96 hours is calculated as lactate production/glucose utilization, and expressed as a percentage. Cumulative leptin production is calculated as the change of media leptin concentrations at 24, 28, 72, and 96 hours, correcting for the amount of leptin removed during sampling. The area under the curve for leptin production between 0-96 hours is calculated by the trapezoidal method. The experimental results from each adipocyte suspension prepared from a single animal are analyzed in relation to a control well from the same suspension. To examine the relationships between glucose uptake, lactate production, glucose conversion to lactate, and leptin secretion, simple and multiple linear regression analyses are performed with a statistics software package (StatView for Macintosh, Abacus Concepts, Inc., Berkeley, Calif.). Data are expressed as means+SEM.

### Example 2

### ADIPOCYTE CULTURE PROTOCOL

Day Before Preparation:

Make phosphate-hepes buffer (instructions on folsh dessicator). Autoclave supplies: Incubation jars (60 ml for rat, 30 ml for mice), filters (400 um for rat, 250 um for mice) long needles (+6), 1 ml pipet tips (+6 boxes), 0.2 ml pipet tips (1 box), surgical equipment (3-5 small scissors, 3 large scissors, 3 forceps), 500 ml reagent jars, 250 and 100 ml reagent jars.

Cut long needle plastic covers to sterilize under uv if needed.

Clear and clean hood, turn on uv light.

Media Preparation:

Place buffer in incubator to warm.

Place 6 ml tubes of FBS, nystatin, penicillin (all in FC freezer) in incubator to thaw.

Get 500 ml bottle of DMEM from walkin cold room (check glucose content).

Place microfuge tube of insulin stock in hood to thaw (−80 freezer, 2^{nd} shelf FC insulin box).

Place microfuge tube of C14 glucose stock in hood to thaw (FC freezer, FC C14 glucose stock).

Turn on hood light in order to turn off uv.

Make basic media by adding 6 ml of FBS, nystatin, penicillin and nonessential amino acids (in FC refrigerator) to 500 ml DMEM.

Make medias (can be the most difficult, intensive, and time-consuming part).

Prepare insulin.

Dilute insulin stock 10×s (0.1 ml to 1.0 ml)

Sterilize with 0.2 um syringe filter.

Label it 160 nM insulin stock.

Dilute 160 nM insulin stock 100×s (0.1 ml to 10 mls).

Label it 1.6 nM insulin stock.

Mix well.

Dilute 1.6 nM insulin stock to 0.48 nM stock and label (1.5 ml to 5 mls).

Dilute 1.6 nM insulin stock to 0.16 nM stock and label (1 ml to 10 mls).

Add the appropriate amount of insulin to medias.

10 microlites of the insulin stock added to 1 ml of media=conc of stock label insulin media (i.e. 100 microliters of 1.6 nM insulin stock to 10 ml of media=1.6 nM insulin media).

Mix medias well, loosen lids, and store in incubator until needed.

Place extra DMEM in incubator until needed.

Prepare for harvesting adipocytes

Cut lab covering for each carcass and label with animal #, absorbent side up.

Label two 60 mm culture dishes with animal # for each animal.

Place 1 dry set by microbalance.

Add buffer to other set.

Place surgical equipment in beaker with 70% EtOH.

Fill a 15 ml labeled conical with buffer.

Label one 10+ ml edta purple top vacutainer with rat #

Set aside lid and place small plastic funnel in tubes.

Get ice for bloods.

Turn on water bath to 37 degrees.

Set up and place FC notebook by microbalance.

Prepare collagenase (5 gram dry-bottle in FC refrigerator).

Rat collagenase concentration=1.25 mg/ml.

Need 2 ml/gram of fat

Need 4 grams of fat/suspension

Therefore for each rat weigh out 10 mg of dry collagenase.

Transfer to 50 ml conical tube.

Add 8 ml buffer/10 mg dry collagenase

(Standard 6 rat recipe=60 mg collagenase/48 ml of buffer)

Mix collagenase well and sterilize with steriflip.

Store in incubator until needed.

Mice collagenase concentration=0.625 mg/ml

Need 2 ml/gram of fat

Assume less than 1 gram of fat/mouse.

Transfer to 50 ml conical tube.

Add 8 ml buffer/5 mg dry collagenase

(Standard 6-10 mouse recipe=12.5 mg collagenase/20 ml of buffer)

Mix collagenase well and sterilize with steriflip.

Store in incubator until needed.

Ready to harvest adipocytes:

Add halothane to harvest adipocytes jar.

Place animal in harvest adipocytes jar.

When unconscious, weigh and record.

Deccapitate, and collect truncal blood in funnel and tube.

Place lid on blood tube, invert, store on ice until centrifuging and separating is possible.

Place animal on harvest adipocytes cloth and take to hood.

Fat Digestion:

Remove epididymal fat pad using buffer-rinsed surgical equipment and place in labeled culture dish with buffer.

Tare dry labeled culture dish with micro balance.

Under hood, transfer epi pad to culture dish using buffer-rinsed forceps.

Weigh and record.

If fat pad weighs more than 4-4.5 grams, remove extra fat using buffer rinsed scissors.

Record suspension fat pad weight on culture dish and in book.

Bring pad back to hood, and re-add buffer.

When all animal fat pads are weighed, add 2 ml of collagenase/gram of fat to labeled suspension jars.

Transfer fat pad to lid of culture dish.

Set timer.

Mince fat for 1-2 minutes (one minute when experienced, two when novice).

Using cell scraper, transfer minced fat to incubation jar.

Set timer and record incubation start time on lid of jar.

Parafilm lid of jar.

Place in 37 degree shaking (motor on 6) water bath for 30 minutes.

Place buffer in incubator

Fat Cleaning:

During incubation prepare for filtration.

For each rat, label a 50 ml conical.

Remove lid and place a 400 um filter on top of tube.

Use a 25 ml pipet to force filter into tube.

For each mouse label a 15 ml conical.

Remove lid and place a 250 um filter on top of tube.

Use a 10 ml pipet to force filter into tube.

At 30 minute incubation (+/− only 1 minute) remove suspension jar from bath.

Add 24 ml buffer (10 ml for mice) and pipet up and down 4 times to mix. Transfer suspension to filter, making sure pipet is in filter, not in a fold.

Allow suspension to drain.

Making sure gloved hands are sterile, scrape filter into conical tube.

Add buffer up to 40 mls (14 ml for mice).

Centrifuge at 1000-1100 rpms—check setting—for 6 minutes.

During centrifuging prepare syringes for cleaning steps.

For each animal label a 20 ml syringe.

Place a long autoclaved needle on syringe.

Place a plastic cover on needle.

Place syringes with needles upright—3 to a 600 ml beaker—to keep sterile.

Label a 600 ml beaker for waste.

At end of centrifuge remove the buffer from underneath cell layer with needle and syringe.

Place this buffer in waste beaker.

Add fresh buffer to 25-35 ml (10-14 ml for mice) depending on quantity available.

Centrifuge at 1000-1100 rpms for 6 minutes.

At end of centrifuge remove buffer and replace with 8-10 ml basic media.

Transfer to labeled 15 ml conical by pouring.

Centrifuge at 1000-1100 rpms for 6 minutes.

At end of centrifuge remove media and add fresh up to no more than 14 ml.

Place in incubator and start a timer.

Incubate for at least 30 minutes, but less than an hour.

Plating in 6 well plates and oxidation vials:

During incubation prepare collagens

Calculate the amount needed figuring 0.5 ml/well and 0.3 ml/oxidation vial plus an extra 3-5 ml.

Transfer that amount to an appropriate-size sterile container (15 or 50 ml conical, 100 ml reagent bottle, or collagen bottle). To minimize collagen waste, pouring is better than pipeting.

Add 1 ml 10× DMEM (50 ml conical tubes in door of FC refrigerator) per 10 ml collagen.

Added 10 M NaOH to collagen to get pH=7, using a red color to judge (not orange, not pink).

It is usually safe to add 0.5% initially (50 ul/10 ml collagen), but collagen can vary by lot and this “safe” quantity can change.

After initial 0.5%, added NaOH only 1-5 ul at a time.

Try not to overshoot since this seems to affect ability of collagen to set.

Try not to take too long, as the collagen can start setting during this process.

When regular collagen is red and ready, add C14 glucose to the appropriate amounts at 1 ul/ml for lipogenesis work (label 1× collagen), and at 3 ul/ml for oxidation work (label 3× collagen).

Set up for collagen pipeting by having a 1 ml pipet for each type of collagen (usually 3—for regular, 1×, and 3×).

Have a 50 ml conical tube labeled to hold each pipets and keep sterile (minimizes the need for fresh tips with each pipeting).

Prepare plating plan based on amount of fat in suspensions, and culture objectives and priorities.

At end of incubation and when collagen, vials, plates are ready, prepare susp 1 for plating.

Remove media to a 2 fat to 1 media ratio.

Use an accurate 200 ul pipet with a sterile wide-open tip for fat pipeting.

To conserve fat, try to complete all pipeting from a single suspension using the same tip.

Mix initially by inverting and then with pipeting, such that suspension is homogenous immediately before each well and vial is plated.

Collagen pipetor person places 0.5 ml collagen in a well, or 0.3 ml collagen in a vial.

Fat pipetor person adds 150 ul of fat suspension directly on collagen.

Plates are gently moved in a circular motion on level surface to spread collagen over entire surface.

Place plated plates and vials in incubator immediately.

Collagen in vials must be in contact with metal shelf to set (use a vial separator insert to avoid tipping).

Finish the plating for all suspensions.

When plating is finished and collagen has set, add 2 ml of appropriate media to each well and label.

Make sure each plate is labeled with FC # and return to incubator.

Place vials by suspension in styrofoam 50 ml conical racks and label (FC # too).

Add 2 ml of media without touching inside of vial.

Return vials to incubator.

While vials are still in incubator, set rubber stopper (with wells and Whatman 1 filter strips) on oxidation vials only 6-10 at a time, when the incubator CO2 is no less than 5%.

Remove rack of vials from incubator and using 2 people, secure rubber stoppers.

Media must not touch wells and paper strips.

Suspension for sizing and lipid measurement:

There must be at least one well/suspension earmarked for sizing and lipid measurement on regular collagen.

Add 2 ml basic media to these wells.

Take 3 Image Pro pictures of each suspension.

Between each suspension take a picture of the suspension #, using the numbered culture lid.

When pictures are taken, aspirate off the 2 ml of media removing as much of the extracellular lipid as possible.

Add 4 ml of methanol to each well.

Parafilm the plate, and replace lid.

Place in refrigerator, making sure each plate is labeled with FC #.

End of 0 hour day.

### Examples 3 and 4

### LEPTIN PRODUCTION ENHANCED VIA NUCLEOTIDE SEQUENCES

### Methods and Materials

Identification and synthesis of PDH-K active site antisense oligonucleotide candidates and nonsense oligonucleotide: The 5 prime end of the PDH-K gene was targeted for possible active site sequences. Net Primer 3 and other similar computer modules was used to confirm and disqualify candidates as primers, based on melting point, % GC content, and tertiary structure. Candidate primers were identified or disqualified as a consensus sequences, common to several species, using the NIH BLAST data-base. Candidate sequences for the nonsense oligonucleotide were screened using computer models for confirmation as primer candidate. The NIH BLAST data-base was used to screen candidate nonsense primers as unrelated to metabolic activity. Both oligonucleotides were synthesized by the Molecular Structure Facility of the University of California, Davis.

Transfection of isolated adipocytes with PDH-K active site antisense oligonucleotide and nonsense oligonucleotide sequences: Oligonucleotides were diluted 8μg/1001μl DMEM. Polyethyleninime (PEI; Aldrich) was diluted 8μg/2001μl DMEM in polystyrene tubes. Diluted oligonucleotide was added one drop at a time to PEI solution and incubated at room temperature for 15 minutes.

A replication-deficient adenovirus was used to assist the transfer of the antisense and “nonsense” oligonucelotides into the cultured adipocytes. Replication-deficient adenovirus (5 dI-342) stock was diluted 2μl/200μl DMEM and then added to PEI-oligonucleotide mixture. After 10 minutes of incubation, 250 μl of each Adenovirus/PEI/ oligonucleotide mixture were added to duplicate wells of 100 μl of adipocyte suspension. Cells were incubated with mixture for 45 minutes. Transfection media was removed, cells were washed one time, and them 0.3 ml of liquid Matrigel matrix was added. 2 ml of 0.48 nM insulin media were added after Matrigel was set and cells were culture for 96 hours.

Measurement of β-galactosidase activity: At 96 hours, media was removed and the cells were washed 2 time in PBS. 0.4 mls of reporter lysis buffer (Promega) was added to each well and incubated for 15 minutes. Cells and buffer were transferred to microfage tubes, vortexed, sonicated for 1 second, and centrifuged for 2 min at 12,000 RPM. Lysate was removed and assayed for β-galactosidase activity using Promega β-Galactosidase Enzyme Assay System.

Transfection of isolated adipocytes with adenovirus-malic enzyme construct: Adenovirus-malic enzyme and adenovirus β-galactosidase constructs were obtained. Isolated cells were plated on collagen as previously described. Cells were incubated in transfection media containing adenovirus stock at 37° C. for 24 hours. Media was removed and cells were washed with PBS. Two ml 0.48 nM insulin media was added and cells were cultured for 96 h.

### Example 3

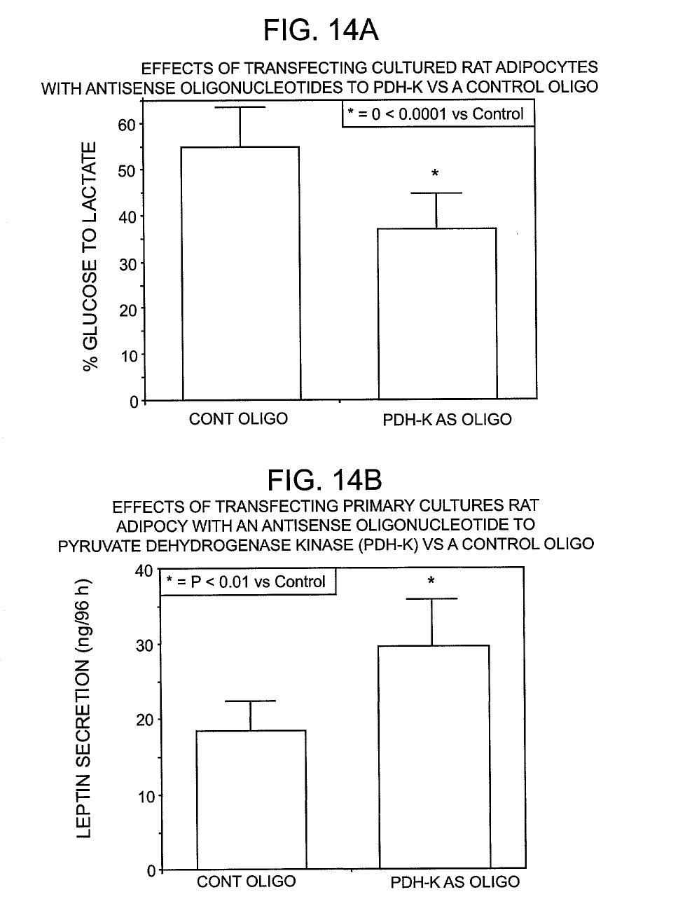

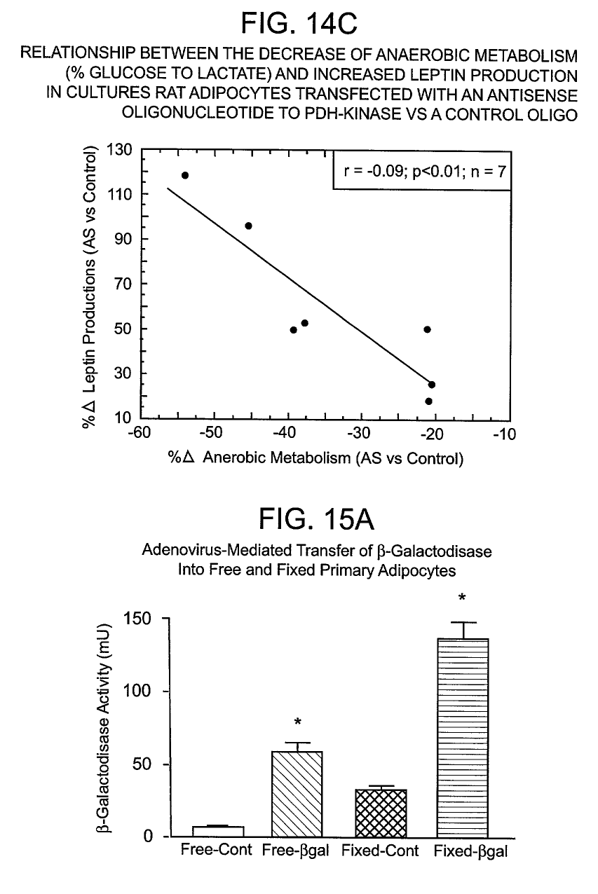

Primary adipocyte cells in cultures were transfected with an oligonucleotide designed to have an antisense sequence to DNA coding for PDH-K. An adenovirus assisted DNA transfer method was used to translocate the antisense oligonucleotide into cultured adipocytes. In these cells (n=7 independent experiments) we observed a substantial decrease of anaerobic glucose metabolism indicated by a highly significant reduction in the proportion of glucose metabolized to lactate (FIG. 14A). In the same cultures, leptin production was markedly increased by an average 82±22% (p<0.01) compared to cells transfected with a control “nonsense” oligonucleotide (FIG. 14B).

The proportional decrease of anaerobic metabolism was highly predictive (r=0.90, p<0.01) of increase of the leptin secretion also observed in this experiment (FIG. 14C). This experiment corroborates the biochemical studies with PDH-K inhibitors and further indicate that a molecular antisense approach is another pharmacological option for increasing endogenous leptin production in vivo. Antisense technology to inactivate specific targets has been suggested in the treatment of a number of diseases including cardiovascular disease (Y1a-Herttuala and Lancet. 355: 213-222, 2000), hypertension (Metcalfe et la, Curr Hypertens Rep. 4: 25-31, 2002), and diabetic vascular disease (Serri and Renier, Metabolism. 44: 83-90, 1995).

### Example 4

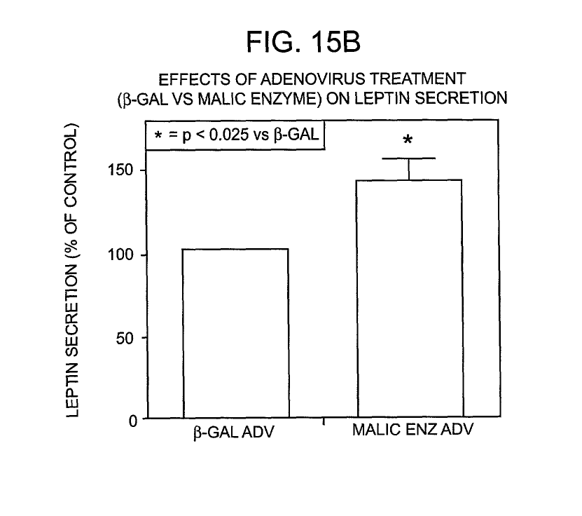

Adipocytes were incubated with a control (β-Galactosidase) engineered adenovirus and a high degree of transfection was obtained as shown in FIG. 15A. Cultured adipocytes were then transfected with an adenovirus vector engineered to comprise the coding sequences for malic enzyme (MD). Cells incubated with the ME virus secreted 40% more leptin than those incubated with a control (β-Gal) adenovirus (FIG. 15B). This shows that this pathway is involved in the metabolic regulation of leptin production. Furthermore, this experiment shows that a gene therapy approach for increasing leptin production is a useful method for increasing endogenous leptin production in vivo in the well and transfer into a 50 ml glass tube. Rinse the well and scrape again in methanol to assure complete transfer of cells. Total triglycerides will be extracted using the Folch method (Folch, 1957). An aliquot of the lipid extract will be placed into vials containing scintillation fluid then radioactivity will be measured.

The first measurement is used to calculate the amount of glucose incorporated into triglycerides. Another aliquot of the lipid extract is placed into pre-weighed aluminum pans to determine the total amount of triglyceride per well. The remaining lipid is saponified and acidified to separate the glycerol and fatty acids. An aliquot of the lipid extract is placed into vials containing scintillation fluid and counted. This second count represents the ¹⁴C-glucose incorporation into fatty acids. By subtraction, the amount that was incorporated into triglyceride though glycerol is also determined. Glucose incorporation into triglyceride and into the fatty acid portion of the triglyceride are calculated by multiplying disintegration per min by total ug of glucose/well over the total DPM/well.

Substrate Oxidation: Oxidation is measured using a modification of the method of Rodbell (Rodbell, 1964) and a modification of the cell culture system described by Bottcher and Furst (Bottcher, 1996). Briefly, adipocytes are isolated, counted and sized as previously described. Adipocytes are plated as described except they are placed in a sterile 20 ml scintillation vial instead of a well. Two ml of treatment media containing [U-¹⁴C]-substrate (0.3 uCi/ml; glucose, fatty acids, malate, fumarate, pyruvate) is added to the vials. The vials are filled with 95%O₂-5%CO₂ gas and capped with rubber stoppers fitted with a hanging center well. Each well contains a 2×8 cm strip of Whatman No. 1 paper. Vials are maintained at 37° C. for 48 hr. After 48 hr, a sample of media is removed from each vial using a 4 inch, 23 gauge needle. Using another syringe and 23 gauge needle, 200 ul of sodium benzethonium is placed onto the paper strip and hanging well to capture CO₂. Concentrated sulfuric acid is added to the vial in order to lyse cells and liberate all CO₂ from the collagen matrix. After 24 hours, the hanging well and paper are transferred to another vial containing scintillation fluid and counted. The data are expressed as % DPM recovered as treatment of obesity and other conditions in which increased leptin production would be beneficial.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of enhancing leptin production, comprising the steps of:
contacting cells with a compound which inhibits pyruvate dehydrogenase kinase; and
allowing the compound to remain in contact with the cells for a period of time and under conditions such that activity of PDHK in the cells is inhibited thereby enhancing production of leptin in the cells.

2. The method of claim 1, wherein the compound is selected from the group consisting of 5,5′-Dithiobis(2-nitrobenzoate)(DTNB), Dichloroacetate (DCA), and N-ethylmaleimide (NEM).

3. The method of claim 1, wherein the cells are cultured mammalian cells.

4. The method of claim 1, wherein the cells are mammalian cells.

5. The method of claim 4, wherein the cells are present in a mammal.

6. The method of claim 5, wherein the mammal is a human.

7. The method of claim 1, wherein the compound is an antisense sequence which reduces PDH-K production in the cells to a level below the level of production prior to contacting the cells with the antisense sequence.

8. A method of enhancing leptin production, comprising the steps of:
contacting cells with an exogenous nucleotide sequences encoding malic enzyme;
allowing the cells to be transfected with the nucleotide sequences in a manner such that malic enzyme is expressed and leptin production of the cells is enhanced.

9. The method of claim 8, wherein the cells are mammalian cells.

10. The method of claim 8, wherein the cells are present in a mammal.

11. The method of claim 10, wherein the mammal is a human.

12. The method of claim 11, wherein the compound is an antisense sequence which reduces PDH-K production in the cells to a level below the level of production prior to contacting the cells with the antisense sequence.

13. A method of causing adipocytes to enhance leptin production, comprising
contacting adipocytes with a pyruviate dehydrogenase kinase (PDHK) inhibitor for a period of time and under conditions such that PDHK is inhibited and leptin production is enhanced.

14. The method of claim 13, wherein the adipocyte is maintained at a temperature in a range of from about 35° C. to about 40° C. at atmospheric pressure ±10%.

15. The method of claim 13, wherein the adipocytes are present in a mammal.

16. The method of claim 15, wherein the mammal is a human.

17. The method of claim 14, further comprising:
transfecting the adipocyte with an antisense sequence which reduces PDHK production.

18. The method of claim 14, further comprising:
transfecting the adipocyte with a sequence encoding malic enzyme.
